(19) United States
(12) Patent Application Publication
(10) Pub. No.: US 2026/0042998 A1
(43) Pub. Date: Feb. 12, 2026

(54) Glyco-Engineered Yeast for Therapeutic Protein Production

(71) Applicants: VIB VZW, Gent (BE); Universiteit Gent, Gent (BE)

(72) Inventors: Nico Callewaert, Nevele (BE); Chiara Lonigro, Gent (BE); Edwin Alexander Rodriguez López, Gent (BE)

(21) Appl. No.: 19/102,943

(22) PCT Filed: Aug. 11, 2022
(86) PCT No.: PCT/EP2022/072531

§ 371 Date: Feb. 11, 2025

(51) Int. Cl. C12N 1/16; C12N 9/10; C12P 21/00; C12R 1/84

(52) U.S. Cl. CPC C12N 1/16; C12N 9/1051; C12P 21/005; C12R 2001/84; C12Y 204/01232

(57) ABSTRACT

The invention relates to the field of glycoprotein production means and methods. More specifically, the present invention provides for a novel yeast strain with a mutant Och1 gene resulting in modified N-glycosylation properties of said strain. Even more specifically, said mutant Och1 gene comprises a sequence coding for a mutant OCH1 protein in which one amino acid near the catalytic site, corresponding to position 151 of the OCH1 protein of the methylotrophic wild type Pichia pastoris strain, is deleted. More specifically, the use of said mutant yeast strain for recombinant expression of glycoproteins results in more homogenous mammalian-like N-glycan structures on said heterologously produced glycoproteins.

### CROSS-REFERENCE TO RELATED APPLICATIONS

This application is a national phase entry under 35 U.S.C. § 371 of International Patent Application PCT/EP2022/072531, filed Aug. 11, 2022, designating the United States of America and published in English as International Patent Publication WO2024/032891 on Feb. 15, 2024, the entireties of which are hereby incorporated by reference.

### INCORPORATION BY REFERENCE

The ST.26 XML Sequence listing named “10488-10878-US-Sequence Listing ST26”, created on Aug. 11, 2022, and having a size of 16,384 bytes, is hereby incorporated herein by this reference in its entirety.

### FIELD OF THE INVENTION

The invention relates to the field of glycoprotein production means and methods. More specifically, the present invention provides for a novel yeast strain with a mutant Och1 gene resulting in modified N-glycosylation properties of said strain. Even more specifically, said mutant Och1 gene comprises a sequence coding for a mutant OCH1 protein in which one amino acid near the catalytic site, corresponding to position 151 of the OCH1 protein of the methylotrophic wild type Pichia pastoris strain, is deleted. More specifically, the use of said mutant yeast strain for recombinant expression of glycoproteins results in more homogenous mammalian-like N-glycan structures on said heterologously produced glycoproteins.

### INTRODUCTION

Manufacturing of therapeutic proteins or biologicals, in general requires a production host capable of providing a protein structure similar to the human protein form, while post-translational modifications, such a N-glycosylation changes in respect to wild-type human protein are often associated with adverse effects (e.g. Dicker, and Strasser, Expert Opin Biol Ther, 2015. 15(10°: p. 1501-16°. Next to biopharmaceuticals, production of a number of enzymes especially for the food and feed sector, may also benefit for the large scale production from alternative (glycol-° engineered host strains.

Glycoprotein production in a (conventional° mammalian system may allow for robust and stable human-like product, though yeast strains, such as the Komagataella phaffii (syn. Pichia pastoris° are becoming a more preferred host for production of biopharmaceuticals because yeast hosts provide for post-translational modifications and protein secretion, and as microbe, showing a faster growth to high cell density in chemically defined media. Unfortunately, the yeast glycosylation pathway produces glycoproteins modified with yeast-specific high-mannose oligosaccharide glycans rather than mammalian complex glycans, which may be immunogenic and lead to rapid clearance. Different efforts have been done to modify the yeast N-glycosylation machinery to produce proteins carrying human-like N-glycans.

One of the main strategies is the knock-out of the Och1 gene, which encodes the Golgi α-1,6-mannosyltransferase that is in charge of transferring the first α-1,6-linked mannose required for further hypermannosylation in yeast. Knock-out of the OCH1 gene results in glycoproteins carrying a majority of Man₈GlcNAc₂ glycans, but also often leads to cells with reduced cell growth, impaired cell wall synthesis and increased temperature sensitivity. To overcome the adverse effect on the yeast viability, different engineering efforts have previously resulted in a number of modified Och1 alleles (FIG. 1°. Several glycoengineered strains able to produce human-type glycosylation have been reported in the literature, though still leaving room for improvements, since yeast-type N- and/or O-glycans are still detected, even if low abundant (Jacobs, et al. Nat Protoc. 4 (2009° 58-70; Nett, et al. Yeast. 28 (2011° 237-252°. Thus, it is necessary to improve the yeast N-glycosylation pattern, to obtain a more suitable glycoprotein for therapeutic purposes while preserving easy handling and scaling up.

### SUMMARY OF THE INVENTION

The present invention reveals a novel yeast strain deficient in OCH1 catalytic activity, wherein the Och1 allele has a 3 base pair nucleotide deletion, resulting in OCH1 mutant protein with a deletion of the glutamic acid at position 151 of the wild type OCH1 as presented in SEQ ID NO:1, called OCH1_E151del herein. Said deletion mutation is located near to the active site, so catalytic activity is affected as such that hypermannosylation of Man₈GlcNAc₂ N-glycans can be prevented using this strain for production of glycoproteins. While it is often observed that inactivation of OCH1 has adverse effects on yeast growth and viability, the mutant yeast strain described herein only showed a severity of the growth delay and temperature sensibility which was somehow attenuated but very close to wild type yeast growth, thus providing for a valid alternative as host for manufacturing of glycoproteins with mammalian-like N-glycan structures.

The first aspect of the invention relates to a yeast strain, comprising a mutant Och1 gene, with a sequence that codes for an OCH1 α-1,6-mannosyltransferase mutant protein lacking the amino acid at position 151 of the wild type OCH1 of SEQ ID NO:1, which is in this case a deletion of a Glutamic acid, or with a sequence that codes for an OCH1 α-1,6-mannosyltransferase mutant protein lacking the corresponding amino acid of a wild type OCH1 homologue of SEQ ID NO:1, wherein said strain is deficient in OCH1 α-1,6-mannosyltransferase activity, and/or produces substantially homogenous N-glycans. In an alternative embodiment, said yeast strain, comprises a mutation in the wild type Och1 gene, with a sequence that codes for an OCH1 α-1,6-mannosyltransferase mutant protein lacking the amino acid at position 151 of the wild type OCH1 of SEQ ID NO:1, or of the corresponding amino acid of a wild type OCH1 homologue of SEQ ID NO:1, wherein said OCH1mutant protein is catalytically inactive, and/or produces substantially homogenous N-glycans. In a further embodiment, said yeast strain comprises a mutant Och1 gene of which the sequence codes for SEQ ID NO:2, or for a homologous OCH1 protein with at least 90% identity thereof, containing the corresponding deletion mutation at position 151 of SEQ ID NO:1, or of a wild type OCH1 homologue thereof.

In a specific embodiment, said yeast strain comprises said mutant Och1 gene, wherein said mutant Och1 gene is present on a chromosome of the yeast genome. More specifically, said mutant Och1 gene may replace the wild type Och1 gene or allele of said yeast strain at its wild type Och1 locus in the genome of said yeast strain.

In an alternative embodiment, said yeast strain comprises a mutant Och1 gene or coding sequence which is expressed from a vector or plasmid obtained by transforming said yeast strain, this providing for a recombinant yeast strain, and wherein the wild type or endogenous Och1 allele on the chromosome of the yeast genome is disrupted, resulting in a mutant yeast strain which is deficient in wild type OCH1 α-1,6-mannosyltransferase activity.

In a further embodiment, said yeast strain as described herein produces homogeneous N-glycan structures on the recombinantly produced glycoproteins, and/or more specifically, wherein the Man₈GlcNAc₂ N-glycan form presents the majority of N-glycosylations.

In further specific embodiments, said mutant yeast strain as described herein is a methylotrophic yeast, or more specifically is a S. cerevisiae strain or a Pichia strain, even more preferably Komagataella phaffii, or Pichia pastoris, even more specifically, Pichia pastoris NRRL Y-14430.

In a further embodiment, said yeast as described herein may comprise a heterologous nucleic acid molecule or plasmid comprising a nucleic acid sequence coding for and capable of expressing a heterologous glycoprotein.

A further aspect of the invention relates to kits for manufacturing glycoproteins with substantially homogeneous N-glycans with Man₈GlcNAc₂ being the predominant N-glycan form, said kits comprising the yeast strain as described herein, and optionally further components, such as media, plasmids, production requirements, and/or an instruction manual.

Another aspect relates to a manufacturing method for recombinant production of glycoproteins of which the majority is not hypermannosylated in the Golgi apparatus of the yeast cells when cultivated, comprising the steps of
a. transforming a plasmid, vector or nucleotide molecule capable of recombinantly expressing a glycoprotein encoded thereby in cells of the yeast strain described herein, and
b. growing the transformed yeast cells to produce said glycoprotein recombinantly,
c. and optionally, isolate said glycoprotein from the yeast culture, wherein said produced glycoprotein comprise predominantly Man₈GlcNac₂ N-glycans on the N-glycosylation site(s°.

Finally, the invention provides embodiments for the use of the Och1 mutant yeast strain described herein, for obtaining glycoproteins deficient in hypermannosylated N-glycans, i.e. containing Man₈GlcNac₂ N-glycans on the majority of their N-glycosylated site(s°.

### DESCRIPTION OF THE FIGURES

The drawings described are only schematic and are non-limiting. In the drawings, the size of some of the elements may be exaggerated and not drawn on scale for illustrative purposes.

FIG. 1. Overview of the different strategies to knock-out the OCH1 gene in yeast. Above OCH1 WT locus: cytoplasmic tail (amino acid M1-Y21°, membrane anchor (F22-S44° and catalytic domain (S45-D404°. Krainer et al. (Scientific Reports. 3 (2013° srep03279° described a complete knock-out of the OCH1 gene (OCH1KO_Krainer°. In the GlycoSwitch strategy the OCH1 activity is depleted by knock-in of the pGLycoSwitchM5 plasmid, which occurs downstream of the Alanine 155 and inadvertently produce an N-term truncated catalytically active Oh1p (OCH1_GSMan5; Contreras et al., EP 1 294 910 B1°. The SuperMan5 strain (Research Corp Inc° is truncated in the N-terminal sequence necessary to target the Och1p to the Golgi apparatus, while expressing a catalytic domain substantially identical to that of the wild type OCH1 protein. The OCH1_E151del has a single amino acid deletion (E151°, which makes the Och1p catalytically inactive. The OCH1_fullKO has a large deletion that starts 83 bp upstream of the coding sequence, therefore no transcription of the OCH1 gene is expected.

FIG. 2. OCH1 gRNA insertion in pPpT4_pHTXhsCas9 plasmid used for generation OCH1 mutants. Left, PCR confirmation of OCH1 RNA guide insertion; and right, digestion with Bgll. Line 1 contain plasmid digested and Line 2 plasmid without treatment.

FIGS. 3A and 3B. PCR confirmation of OCH1 mutations. FIG. 3A. Amplification using primers 5 and 6 with an expected amplicon of 715 bp. FIG. 3B. Amplification using primers 3 and 4 with an expected amplicon size 1150 bp. wt: wild type Pichia pastoris strain NRRLY-11430; 1: OCH1_1 mutant; 2: OCH1_2 mutant; 3: OCH1_3 mutant; 4: OCH1_4 mutant; Δ: ΔOCH1mutant.

FIGS. 4A and 4B. Sorbitol effect on temperature sensibility of OCH1 mutants. Results from 3-day cultures at 30° C. and 37° C. in YPD plates (FIG. 4A° and YPD plates with sorbitol (YPDS° (FIG. 4B°. A 10-fold serial dilution was performed starting at OD₆₀₀ 2.0.

FIGS. 5A and 5B. Analysis of the N-glycosylations from P. pastoris NRRL Y-11430 strains. FIG. 5A. Results obtained with samples from cultures of the WT strain (NRRLY-11430°. FIG. 5B. Results obtained with the OCH1 mutant strains OCH1_1 and OCH1_2, as well as ΔOCH1. Maltodextrin and N-glycans from bovine RNase B were used as reference.

FIG. 6. Digestion of glycoproteins produced by P. pastoris NRRL Y-11430 strains with α-1,2-mannosidase and Jack Beam Mannosidase. Results from capillary electrophoresis of the pool of proteins produced by the wild type strain (NRRLY-11430° and representative images from the mutant strains ΔOCH1 and OCH1_1.

FIGS. 7A-7C. Differences between the WT and the OCH1 mutant strains. (FIG. 7A° Pichia WT and OCH1_fullKO strains were grown in liquid cultures separately and then mixed in 1:1 ratio based on the OD₆₀₀ before plating. Plates were incubated for 60 hours at 30° C. The WT NRRL Y11430 strain has bigger colonies of smooth and creamy morphology. (FIG. 7B° The OCH1_fullKO has a wrinkled and sandy morphology and colonies grow slower. (FIG. 7C° Pichia supernatant of OCH1_E151del and OCH1fullKO expressing M2eVHH-Fc. The supernatant of the OCH1fullKO contains much more Pichia cell proteins as compared to the OCH1_E151del strain, indicating cell lysis.

FIGS. 8A and 8B. Growth curves of the two OCH1 mutant strains compared to the NRRL Y-11430 WT strain. (FIG. 8A° exponential growth curves were performed in BMGY medium in shake flasks. Each strain was grown in triplicate (except for OCH1_fullKO that was grown in duplicate° and the growth was assessed by measuring OD600 nm. Data points are the mean of three measurement replicates. The curve was fitted using Grofit R-package using a Gompertz fit mathematical model. (FIG. 8B° Growth rates were calculated as the maximum slope of the spline fit (μ°. The error bars represent standard errors.

FIGS. 9A and 9B. N-glycosylation profile of the two OCH1 mutant strains. (FIG. 9A° Comparison of the N-glycosylation profile of (III° the OCH1_fullKO and the (IV° OCH1_E15del strains. (FIG. 9B° Glycans from the (III° OCH1_fullKO were treated with (IV° α1,2 mannosidase and (V° ccMan5 and CIP. Panel (I° and (II° are RNaseB and Dextran respectively.

FIG. 10. GALNS activity of NRRLY-11430 WT and OCH1 mutants. Results from cultures at 2 mL scale. The results are shown as mean±SD, n=3.

FIG. 11. Characterization of N-glycosylation profile of hprGALNS produced in the P. pastoris NRRL Y-11430ΔOCH1 strain. N-glycans, from the purified enzyme, were analyzed by capillary electrophoresis before and after digestion with CcGH92_4 and/or CcGH92_5.

FIGS. 12A and 12B. Differences between the NRRL Y-11430(wild type) and two OCH1 mutant strains. (FIG. 12A° Pichia NRRL Y-11430, OCH1_E151del and OCH1_fulLKO strains on plates. (FIG. 12B° Pichia NRRL Y-11430 and OCH1_fullKO strains were grown in liquid cultures separately and then mixed in 1:1 ratio based on the OD600before plating. Plates were incubated for 60 hours at 30° C. The NRRL Y11430 strain has bigger colonies of smooth and creamy morphology. The OCH1_fullKO has a wrinkled and sandy morphology and colonies grow slower, while the OCH1_E151del is indistinguishable from the NRRLY-11430.

FIGS. 13A and 13B. Mass spectra of thePichia-produced M2eVHH-Fc digested with FabRICATOR and (FIG. 13A) α1,2 mannosidase or (FIG. 13B) Jack Bean α1,2/3/6 mannosidase. (FIG. 13A)Peak 2 M2eVHH-hinge, Peak 1 M2eVHH-hinge minus glycine, Peak 3 M2eVHH-Fc+2× mannoses, Peak 4 aglycosylated Fc, Peak 5 Fc+Man5GlcNAc2glycans, Peak 6 Fc+Man5-8GlcNAc2glycans. (FIG. 13B) Peak 2 M2eVHH-hinge, Peak 1 M2eVHH-hinge minus glycine, Peak 4 aglycosylated Fc, Peak 5 Fc+ManGlcNAc2glycans, Peak 6 Fc+ManGlcNAc2+2×O-mannoses, Peak 7 Fc+Man3-5GlcNAc2glycans.

FIGS. 14A and 14B. Therapeutic administration of M2eVHH-Fc restricts influenza virus replication in mice. (FIG. 14A) Morbidity expressed as % of body weight loss. (FIG. 14B) % of survival of influenza challenged mice.

FIG. 15. Alignment of OCH1 protein sequence from Pichia pastoris and Saccharomyces cerevisiae. In bold underlined, the amino acid at position 151 (Glu, E° of Pichia pastoris OCH1 is indicated, as well as the corresponding amino acid in S. cerevisiae OCH1 (Isoleucine, I°. OCH1-S.cerevisiae (AC: YGL038C, SEQ ID NO: 15° is the sequence from S. cerevisae, OCH1-Pichia pastoris corresponds to SEQ ID NO:1.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s° described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012°; and Ausubel et al., Current Protocols in Molecular Biology (Supplement 114°, John Wiley & Sons, New York (2016°, for definitions and terms of the art. Unless defined otherwise, all technical and scientific terms used herein have the same meaning as commonly understood by one of ordinary skill in the art (e.g. in molecular biology, biochemistry, and structural biology.

“Nucleotide sequence”, “DNA sequence” or “nucleic acid molecule(s°” as used herein refers to a polymeric form of nucleotides of any length, either ribonucleotides or deoxyribonucleotides. This term refers only to the primary structure of the molecule. Thus, this term includes double- and single-stranded DNA, and RNA. It also includes known types of modifications, for example, methylation, “caps” substitution of one or more of the naturally occurring nucleotides with an analog. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to messenger RNA (mRNA°, cDNA, recombinant nucleotide sequences or genomic DNA, while introns may be present as well under certain circumstances. “Gene” as used here includes both the promoter region of the gene as well as the coding sequence. It refers both to the genomic sequence (including possible introns° as well as to the cDNA derived from the spliced messenger, operably linked to a promoter sequence. “Promoter region of a gene” as used here refers to a functional DNA sequence unit that, when operably linked to a coding sequence and possibly placed in the appropriate inducing conditions, is sufficient to promote transcription of said coding sequence. An “allele” is one of two or more versions or variants of a gene, at a given genomic location or locus.

The terms “protein”, “peptide”, and “polypeptide” are interchangeably used further herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation, ubiquitination, sumoylation, and acetylation, among others known in the art. Based on the amino acid sequence and the modifications, the atomic or molecular mass or weight of a polypeptide is expressed in (kilo°dalton ((k°Da°. By “isolated” or “purified” is meant material that is substantially or essentially free from components that normally accompany it in its native state. For example, an “isolated protein”, “isolated nucleic acid molecule”, refers to a protein, or nucleic acid molecule, which has been purified from the molecules which flank it in a naturally-occurring state, or in its production host, e.g., other secretion or ER/Golgi residing proteins or cellularly present nucleic acid molecules, as identified and disclosed herein which have been removed from the molecules present in the sample or mixture, or yeast or cellular environment, such as a production host, that are adjacent to said material, by using the detergents, or other agents, and/or purification means as disclosed herein, and as known in the art. An isolated protein or nucleic acid molecule or composition can be generated by chemical synthesis followed by further treatments or can be generated by recombinant production or by purification from a complex sample.

“Homologue”, “Homologues” of a protein encompass peptides, oligopeptides, polypeptides, proteins and enzymes having amino acid substitutions, deletions and/or insertions relative to the unmodified protein in question and having similar biological and functional activity, or in case of a mutant homologue, a similar deficiency in a certain activity, as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, lie, Phe, Tyr, Trp, Lys, Arg, His, Asp, Glu, Asn, Gln, Cys and Met, also indicated in one-letter code herein° occurs in both sequences to yield the number of matched positions, dividing the number of matched positions by the total number of positions in the window of comparison (i.e., the window size°, and multiplying the result by 100 to yield the percentage of sequence identity. A “corresponding” amino acid, as referred to herein, is meant to provide for the amino acid of a homologous protein that corresponds to the same position (and function° of the amino acid of a wild type or reference protein as determined by aligning one or more protein sequences using a pairwise or multiple alignment tool, as known to the skilled person. Theoretically, when for instance two protein sequences are aligned, the amino acids which are structurally and functionally similar will be corresponding in their position in the alignment. A “substitution”, or “mutation”, or “variant” as used herein, results from the replacement of one or more amino acids or nucleotides by different amino acids or nucleotides, respectively as compared to an amino acid sequence or nucleotide sequence of a parental or reference protein or a fragment thereof. Similarly, a deletion mutant as used herein refers to the resulting protein or nucleic acid molecule wherein one or more amino acids or nucleotides, resp., have been removed as compared to an amino acid or nucleotide sequence of a parental or reference sequence, or fragment thereof. It is understood that a protein or a fragment thereof may have conservative amino acid substitutions which have substantially no effect on the protein's activity.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g. codon-optimized°, post-translational modifications and/or functional properties (i.e., altered characteristics° when compared to the wild-type gene or gene product. It is noted that naturally occurring mutants can be isolated; these are identified by the fact that they have altered characteristics when compared to the wild-type gene or gene product.

### DETAILED DESCRIPTION

The present invention is based on the serendipitous finding of a 3 bp deleted alpha-1,6-mannosyltransferase Och1 gene resulting in an altered protein sequence of the Pichia pastoris NRRL Y-14430 OCH1 amino acid sequence (SEQ ID NO:1° close to its catalytic site, specifically a deletion of the glutamic acid at position 151, resulting in the mutant OCH1 protein corresponding to SEQ ID NO:2, as described herein. The presence of said genomic mutant Och1, introduced using a CRISPR/Cas editing strategy, revealed to provide for a yeast strain allowing recombinant production of glycoproteins with predominantly Man₈GlcNac₂ N-glycans, and thus deficient in the hypermannosylation normally occurring in the strain with wild type Och1, with a growth and phenotypic characteristics close to the wild type strain. The present invention thus provides for a novel engineered yeast host system, more specifically Pichia pastoris NRRL Y-14430 OCH1_E151del as named herein, suitable for stable and efficient manufacturing of therapeutic proteins or biopharmaceuticals, such as antibodies, or for instance VHH-Fc fusions, as exemplified herein, as well as enzymes or further protein classes for which glyco-engineered yeast production hosts may be advantageous.

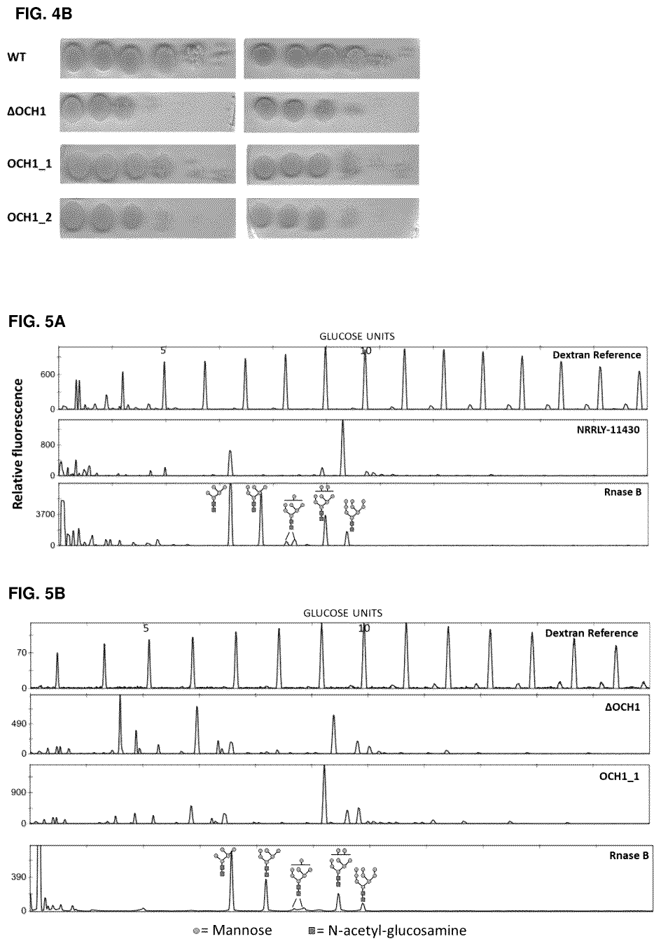

Yeast as Host for Recombinant Protein Production Yeasts are eukaryotic single-cell microorganisms that are easy to grow and therefore one of the most important model organisms for molecular biology and genetics. The genome of the baker's yeast S. cerevisiae was the first eukaryotic genome to be sequenced in 1996. In the meantime, many other yeasts started to be investigated. The present invention specifically relates to a glycol-engineered yeast strain, wherein the presence of an Och1 mutant gene affects the N-glycosylation pathway. The mutant OCH1 protein as described herein for Pichia pastoris may in the scope of the present invention also be obtained in other yeast species, since the region near the catalytic site of the OCH1 enzyme is relatively conserved, as shown for instance for Y. lipolytica, S. cerevisiae, C. albicans and Sch. Pombe OCH1 in the alignment of OCH1 amino-acid sequences shown in FIG. 4 of Barney-Verdier et al. (Microbiology (2004°, 150, 2185-2195°. Pichia pastoris belongs to the methylotrophic yeasts, providing for further advantages. Methyloptrophic yeast (belonging to Hansenula, Candida, Pichia, Torulopsis genera° indeed are capable to metabolise monocarbonic compounds like methanol and formaldehyde (Pawel, et al., Biodegradation 12, pp: 169-177, 2001; Negruta et al., Romanian Biotechnological Letters Vol. 15, No. 4, p5369, 2010°, and appear in nature in particular in moulds, fruit and other vegetable products, exudates of trees and their barks (Craveri et al. Taxonomical examination and characterization of a methanol-utilizing yeast, Antonie van Leeuwenhoek 42, pp: 533-540, 1976°. Methylotrophic yeast are Crabtree negative organisms that do not produce ethanol in anaerobic conditions, hence can grow to extremely high cell densities, which often translates into high product yield. In addition, they can efficiently secrete high molecular weight proteins, which in S. cerevisiae, would be retained in the periplasmic space. Methylotrophic yeast strains which can be modified using the present methods include but are not limited to yeast capable of growth on methanol such as yeasts of the genera Candida, Hansenula, Torulopsis, and Pichia. A list of species which are exemplary of this class of yeasts can be found in C. Anthony (1982°. The Biochemistry of Methylotrophs, 269. Pichia pastoris, Pichia methanolica, Pichia anomola, Hansenula polymorpha and Candida boidinii are examples of methylotrophic yeasts useful in the practice of the present invention. Preferred methylotrophic yeasts are of the genus Pichia. Especially preferred are Pichia pastoris strains GS115 (NRRL Y-_15851°; GS190 (NRRL Y-_18014° disclosed in U.S. Pat. No. 4,818,700; PPF1 (NRRL Y-_18017° disclosed in U.S. Pat. No. 4,812,405; PPY120H and yGC4; as well as strains derived therefrom.

In particular, Pichia pastoris, which was recently reclassified as Komagataella phaffii [1], but hereinafter still referred colloquially to as Pichia, is now one of the preferred hosts of the biotechnological sector for recombinant protein production. Pichia pastoris triggered the interest to be used as a cheap single cell protein source for animal feed, because of its capacity of growing to extremely high cell densities (130-150 dry cell weight/1° using methanol as its sole carbon source and metabolic energy [2], and its potential of becoming an excellent organism for heterologous protein expression [3], became also clear from further developments, such as a commercial Pichia expression system (Invitrogen Corporation (USA°°, which is currently used worldwide [4]. The original Pichia strain was deposited as a patent deposition, in two culture collection entries, NRRL Y-11430 and CBS7435 [5]. The commonly used GS115 strain is an auxotrophic his4 strain derived from CBS7435 by random mutagenesis with N-methyl-N′-nitro-N-nitrosoguanidine (NTG°.

The methanol utilization pathway (MUT° of P. pastoris and other methylotrophic yeasts has been deeply characterized and reviewed [6]. The first part of the MUT pathway takes place in specialized organelles called peroxisomes that can take up 80% of the cytoplasmic space during methanol induction. The first step in the MUT pathway is catalyzed by alcohol oxidase that oxidizes methanol to formaldehyde and hydrogen peroxide. Hydrogen peroxide (H₂O₂° is detoxified by a catalase (CAT°, while formaldehyde can be either oxidized to carbon dioxide in the dissimilative pathway or assimilated via the C1-assimilation pathway. In the dissimilative pathway, formaldehyde reacts with glutathione (GS°, upon which it is oxidized by two consecutive reactions to CO₂, resulting in the regeneration of GSH and production of NADH. In the assimilation pathway, the transketolases DAS1 and DAS2 condense formaldehyde with xylulose-5-phosphate to form dihydroxyacetone (DHA° and glyceraldehyde-3-phosphate (GAP°, which are then exported to the cytosol where they are further metabolized during gluconeogenesis and pentose phosphate cycle reactions, yielding a production of 1 mole of GAP per 3 moles of methanol entering the assimilative pathway. The MUT pathway is in general repressed by glucose, glycerol, and ethanol and induced by methanol. In P. pastoris there are two alcohol oxidase genes, AOX1 and AOX2, of which AOX1 is the major contributor to the MUT. A knock-out of the AOX1 gene results in a strain with a slow-growth phenotype on methanol, called MutS (methanol utilization slow°, while a knock-out of both AOX1 and AOX2 results in a strain with the Mut-phenotype, which is unable to grow on methanol [7].

The Alcohol Oxidase 1 protein (AOX1p° can constitute up to 35% of soluble protein under methanol feeding conditions. Its tightly regulated promoter (PAOX1° has been successfully used as a strong inducible promoter for recombinant protein expression and the first industrial-scale production of hydroxynitrile lyase was established with an extraordinary yield of 20 g/l [3,8,9].

### Glycoprotein Production in Yeast

As yeasts are eukaryote, they can perform post-translational modifications, including glycosylation. Protein glycosylation can have a strong impact on protein folding, stability, and activity, and therefore, it is a critical attribute for production of biopharmaceuticals where it influences the drug pharmacokinetics, pharmacodynamics, and immunogenic properties. Two main types of glycosylation occur in eukaryotes: N- and O-glycosylation. N-glycosylation occurs at the Asparagine in the consensus sequence N-X-S/T (where X is any amino acid but proline°, while there is no consensus sequence for O-glycosylation, which is likely mostly driven by protein conformation and occurs on Ser/Thr residues. The yeast N-glycosylation pathway produces glycoproteins modified with high-mannose oligosaccharide glycans rather than mammalian complex-type glycans. Similar to other eukaryotes, P. pastoris performs a complex glycosylation pathway that starts in the endoplasmic reticulum with the synthesis of a lipid linked oligosaccharide by stepwise addition of nucleotide activated monosaccharides in the cytosol side of the ER-membrane that will be then translocated to the ER lumen where final extension is achieved to obtain a final Glc₃Man₈GlcNAc₂ that will be finally transferred to the polypeptide chain. Afterwards the protein is transferred to Golgi apparatus where further modifications of the glycan chain occur [14,15]. The specific machinery for such modifications accounts for the differences observed among eukaryotic organisms [15]. In higher eukaryotes, the Man8GlcNAc2 is further processed to Man5GlcNAc2 by the Golgi resident Mannosidase I, and the first α1,2 GlcNAc is added, followed by removal of 2 mannoses by Mannosidase II and decorated with an additional N-acetylglucosamine (GlcNAc°, galactoses (Gal°, sialic acids (Sia°, and fucoses. In yeast, the Man8GlcNAc2 is the substrate of the α1,6 mannosyltransferase OCH1, which initiates the elongation of the α1,6-linked mannose chain [18]. The addition of the α1,6 branch is necessary to create the substrate of the downstream-acting α1,2 mannosyltransferases. So different to human cells, which synthetize complex glycans by addition of different monosaccharides and sialic acid, yeast glycosylates its proteins with high mannose carbohydrates, and P. pastoris in particular, expresses mainly glucosyltransferases involved in the synthesis of oligomannose type glycans [16, 17]. Therefore, similar to other yeasts, in P. pastoris the activity of the OCH1 protein, an α-1,6-mannosyltransferase, is important for the addition of a mannose by α-1,6 linkages [14-16]. Then different mannosyltransferases will extend the α-1,6 chain, that will be substrate for medial- and trans-Golgi-residing α-1,2-mannosyltransferases. However different to other yeast like S. cereviseae, it has been reported that P. pastoris lacks of several of these enzymes therefore displaying shorter glycans without terminal addition of α-1,3-mannose residues [14, 15], which makes the latter the preferred host for recombinant protein expression. Moreover P. pastoris is not able to transfer α1-3 mannose residues to the glycan, which represents an advantage due to the immunogenicity of such structure. In addition, easiness of genetic manipulation with tools already established for the model organism S. cerevisiae made that Pichia became one of the preferred hosts for recombinant protein expression. In the early 2000s, Pichia obtained the GRAS status (Generally Recognized as Safe° for pharmaceutical production, after which the first Pichia-produced recombinant biopharmaceutical product ecallantide (Kalbitor®°, a plasma kallikrein inhibitor, was approved by the FDA in 2009 for the treatment of hereditary angioedema and in the prevention of blood loss in cardiothoracic surgery [10]. Today, there are more than 70 Pichia-produced biopharmaceutical products on the market or in late stage development [11].

Next to biopharmaceuticals, Pichia is used for the production of a number of enzymes especially for the food and feed sector, including phytase, lipase, mannanase and xylanase. [12]. Recently, Pichia was used for the large scale production of leghemoglobin, the soybean heme protein, an essential ingredient of the meat-free Impossible Burger [13].

### OCH1 Glyco-Engineering

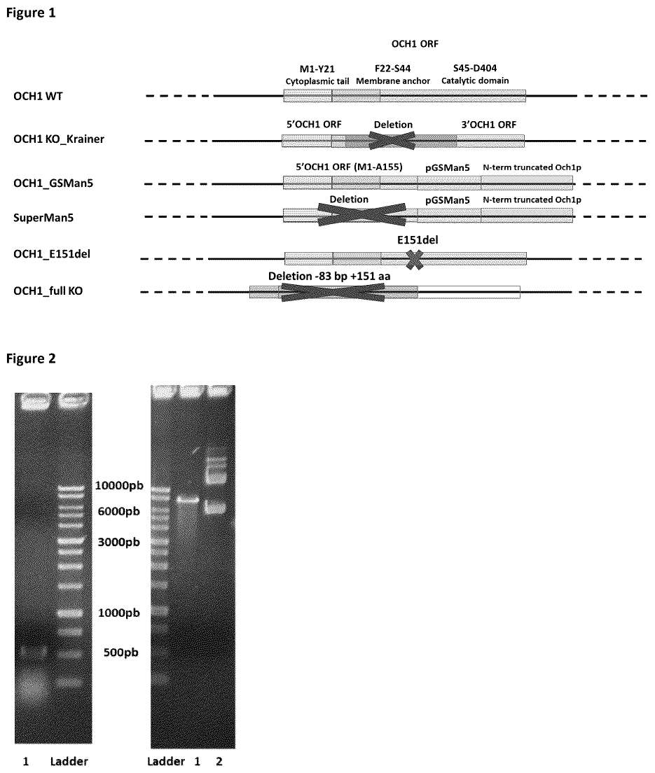

To prevent hypermannosylation and obtain more homogenous glycosylation of the recombinant protein, mainly with Man8GlcNAc2 structure, the OCH1 gene can be knocked-out. This absence of hypermannosylation is favorable for protein characterization and downstream purification processes. Unfortunately, the och1-strain shows an impaired phenotype, including slow growth, abnormal colony shape, and temperature sensitivity, due to rearrangements of cell wall components [19,20]. Because this is the first step towards the humanization of the yeast glycans and in general used to obtain homogeneously glycosylated glycoproteins, solutions to rescue the growth defect of the och1-strain have been sought for (FIG. 1°. In the GlycoSwitch strategy, the disruption of the OCH1 gene is obtained via a knockin strategy, which also facilitates the generation of the strain because of the particularly low efficiency of double homologous recombination in this locus. The construct is designed to express a non-catalytically active OCH1 protein (Met1-Ala155° and a promoterless second copy with in frame stop codons upstream of the ORF, and at the same time knocks-in the second step of glycan humanization, the ER-retained T. reesei α-1,2-mannosidase-HDEL, which results in secreted glycoproteins decorated mainly with Man5GlcNAc2. However, when genome sequencing became available, it was found that this GlycoSwitchM5 strain had an alteration in the design of the knock-in vector: the second copy of the OCH1 ORF was of such nature that an N-term truncated product could still be formed. Likely because of the presence of this low amount of truncated Och1p, the loss of strain vitality associated with full OCH1 knock-out is mitigated. Contrary to what was observed in the full-knockout strain described by Krainer et al., this glycoengineered strain shows a similar doubling time as the wild type strain, but with a somewhat lower cell density at stationary phase [21]. The commercially available SuperMan5 strain has additional deletion of the OCH1 N-terminus cytoplasmic domain that enhances the glycoengineered strain stability [22]. Pekarsky et al. performed a physiological and morphological comparison of the och1-Man8GlcNAc2 strain obtained with the full knock-out of the ORF, and the SuperMan5 strain [23]. The two glycoengineered strains showed similar morphological traits, characterized by cell agglomeration, in which the cells at the interior of the agglomerate were less viable, as compared to a wild type strain. However, the och1° showed reduced growth and productivity both in shake flasks and in a high-cell density fermentation as compared to the SuperMan5. Interestingly, both glycoengineered strains showed a better conversion of substrate to biomass, which possibly led to a higher productivity for the SuperMan5 strain as compared to the wild type strain. The reduced fitness of the och1° strain cannot be attributed to the agglomeration behavior, but rather to some sort of stress, likely cell wall stress sensing, which may differ in the two strains because of the different knock-out strategies adopted (knock-in vs. knock-out° [23].

In a first aspect of the invention, a yeast strain is described, comprising a mutant alpha-1,6-mannosyltransferase Och1 gene, characterized in that said mutant Och1 gene encodes the OCH1 α-1,6-mannosyltransferase mutant protein comprising a deletion of the amino acid at position 151 of the wild type OCH1 of SEQ ID NO:1, or a an Och1 gene encodes the OCH1 α-1,6-mannosyltransferase mutant protein comprising a deletion of the corresponding amino acid of a wild type OCH1 homologue of SEQ ID NO:1, upon alignment of the wild type OCH1 sequences thereof, wherein said strain is deficient in OCH1 α-1,6-mannosyltransferase activity. The Och1 mutant yeast strain as referred to herein thus has no or very little OCH1 α-1,6-mannosyltransferase catalytic activity, which means that the activity is not detectable or is less than 50%, or less than 40% or less than 30% or less than 20% or less than 10% or even less than 5% or less than 3% or less then 1% of the activity as determined for the strain with α-1,6-mannosyltransferase activity of the wild type Och1. Said strain is thus deficient in OCH1 α-1,6-mannosyltransferase activity, wherein deficient herein refers to that the enzymatic activity is not detectable or is less than 50%, or less than 40% or less than 30% or less than 20% or less than 10% or even less than 5% or less than 3% or less than 1% of the activity as determined for the strain with α-1,6-mannosyltransferase activity of the wild type Och1. The production of a glycoprotein in said yeast strain thus results in glycoproteins with a substantially homogenous N-glycan structure, preferably predominantly with a Man₈GlCNAc₂ identity. With substantially homogenous herein referring to the observation that the majority of the (heterologously expressed° glycoprotein produced in said yeast strain as described herein contain N-glycans of said Man₈GlCNAc₂ structure, wherein majority means that at least 70% or preferably at least 80%, or at least 85%, or at least 90%, or at least 95% or more preferably at least 97%, or 99% of said glycoproteins contains said N-glycan structure, as determined in the glycosylation profile, for which the methodology are known to the skilled person, and as exemplified herein.

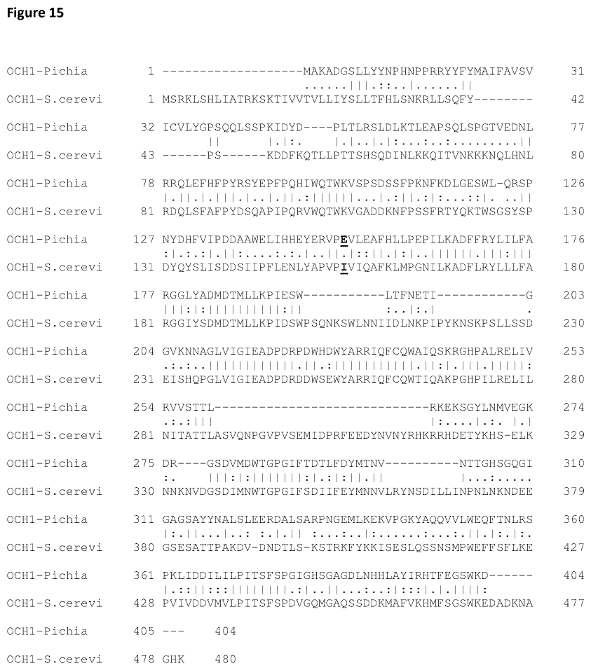

In a specific embodiment, the yeast strain described herein comprises a mutant Och1 gene sequence, which codes for the mutant OCH1 protein of SEQ ID NO:2, which corresponds to the OCH1 protein of Pichia pastoris NRRL Y-14430 as defined by SEQ ID NO:1, but with a deletion of the glutamic acid at position 151 of SEQ ID NO:1, resulting in SEQ ID NO:2. An alternative embodiment, wherein the yeast strain may be of a different identity than Pichia pastoris, relates to said strain comprising a mutant Och1 gene sequence, which codes for the mutant OCH1 protein of the sequence of a homologous mutant OCH1 protein with at least 90% identity to SEQ ID NO:2, wherein the residue of the non-mutated or wild type OCH1 protein form of said OCH1 protein sequence of said yeast strain being different from Pichia pastoris upon alignment with SEQ ID NO:1, is deleted at position 151 in said mutant OCH1 protein with at least 90% identity to SEQ ID NO:2. More specifically, said homologous mutant OCH1 protein may have at least 95% identity to SEQ ID NO:2, 97% identity, or 99% identity to SEQ ID NO:2. One specific embodiment relates to a S. cerevisiae strain comprising a mutant Och1 gene sequence, which codes for the mutant OCH1 protein which corresponds to the protein of SEQ ID NO:15 wherein the Isoleucine(I° at position 155, which is the corresponding amino acid of Glu151 in SEQ ID NO:1, is deleted (also see FIG. 15°.

In further specific embodiments, the yeast strain described herein comprises said mutant Och1 gene as part of a chromosome, i.e. it the mutant is integrated in the genome or is a genomic mutant or variant, defective in OCH1 activity. More preferably, the mutant Och1 gene here may replace the endogenous or wild type Och1 gene at the Och1 locus of said yeast strain.

Alternatively, it is also embodied herein that the yeast strain as described may comprises the mutant Och1 gene comprising said E151 deletion, or corresponding mutation, which is present in the yeast cell on a plasmid or DNA sequence that is not integrated in the genome, and wherein the native or endogenous Och1 gene on the chromosome has been disrupted, so that said strain is deficient in wild type OCH1 α-1,6-mannosyltransferase activity.

The yeast strains as described herein also include those yeast strains which have been genetically engineered to express one or more heterologous glycoproteins of interest. The glycosylation on the heterologous glycoproteins expressed from these previously engineered strains can be reduced by transforming such strains, using yeast transformation protocols as known to the skilled person. A nucleic acid molecule, vector or plasmid, can be introduced into the cells of a yeast strain using known methods such as the spheroplast technique, described by Cregg et al. 1985, or the whole-_cell lithium chloride yeast transformation system, Ito et al. Agric. Biol. Chem. 48:_341, modified for use in Pichia as described in EP 312,934. Other published methods useful for transformation of the plasmids or linear vectors include U.S. Pat. No. 4,929,555; Hinnen et al._Proc. Nat. Acad Sci. USA 75:_1929 (1978°;_Ito et al. J. Bacteriol. 153:_163 (1983°; U.S. Pat. No. 4,879,231; Sreekrishna et al. Gene 59:_115 (1987°. Electroporation and PEG1000 whole cell transformation procedures may also be used. Cregg and Russel Methods in Molecular Biology: Pichia Protocols, Chapter 3, Humana Press, Totowa, N.J., pp. 27-39 (1998°. Transformed yeast cells can be selected by using appropriate techniques including but not limited to culturing auxotrophic cells after transformation in the absence of the biochemical product required (due to the cell's auxotrophy°, selection for and detection of a new phenotype, or culturing in the presence of an antibiotic which is toxic to the yeast in the absence of a resistance gene contained in the transformants. Transformants can also be selected and/or verified by integration of the expression cassette into the genome, which can be assessed by e.g., PCR analysis or sequencing. A further aspect of the invention thus relates to a method for recombinantly producing a glycoprotein with reduced glycosylation, more specifically wherein the majority of its N-glycans being Man₈GlcNac₂, in yeast, comprising the steps of making a transgenic yeast expressing a nucleotide sequence encoding a heterologous glycoprotein wherein said yeast comprises the Oh1 mutant gene to code for the Och1 mutant as described herein, lacking the Glu at position 151 as compared to SEQ ID NO:1, and incubating said transgenic yeast cells to express the glycoprotein recombinantly, and optionally, isolate said glycoprotein from the cultivated yeast.

Alternatively, a yeast strain comprising the mutant Och1 gene as described which has already been genetically engineered to express a glycoprotein is used in the method as described herein. Alternatively, in the embodiment wherein the yeast strain does not express a glycoprotein of interest, and the Och1 wild type gene is disrupted, but does not yet contain the Och1 mutant gene as described herein, a yeast strain can be transformed, either consecutively or simultaneously, with both a nucleotide sequence capable of expressing then glycoprotein and to introduce the plasmid or vector or nucleic acid molecule comprising the mutant Och1 gene.

“A glycoprotein” as used herein refers to a protein which, in yeasts, is either glycosylated on one or more asparagines residues or on one or more serine or threonine residues, or on both asparagines and serine or threonine residues. The term “reduced glycosylation” refers to a reduced size of the carbohydrate moiety on the glycoprotein, particularly with fewer mannose residues, when the glycoprotein is expressed in a yeast strain which has been modified as described herein, as compared to a wild type, unmodified strain of the methylotrophic yeast. In accordance with the present invention, the production of a glycoprotein of interest with reduced glycosylation can be achieved in a number of ways. A nucleotide sequence capable of expressing a glycoprotein can be introduced into a yeast strain which has been previously modified in accordance with the present invention, i.e., a OCH1_E151del mutant strain transformed with one or more of vectors and capable of producing glycoproteins with reduced glycosylation.

Glycoproteins produced can be purified by conventional methods. Purification protocols can be determined by the nature of the specific protein to be purified. Such determination is within the ordinary level of skill in the art. For example, the cell culture medium is separated from the cells and the protein secreted from the cells can be isolated from the medium by routine isolation techniques such as precipitation, immunoadsorption, fractionation or a variety of chromatographic methods.

It is to be understood that although particular embodiments, specific configurations, compositions, as well as materials and/or molecules, have been discussed herein for methods, compositions and products according to the disclosure, various changes or modifications in form and detail may be made without departing from the scope of this invention. The following examples are provided to better illustrate particular embodiments, and they should not be considered limiting the application. The application is limited only by the claims.

### Examples

### INTRODUCTION

The knock-out of the OCH1 gene is the first step towards yeast glycan humanization and it results in glycoproteins carrying a majority of a Man₈GlcNAc₂ glycans. Next to the decreased glycan heterogeneity, the full deletion of Och1p results in cells with reduced cell growth, impaired cell wall synthesis and increased temperature sensitivity (Krainer, et al., Scientific Reports. 3 (2013° srep03279°. In the GlycoSwitch strategy, instead of a deletion, the disruption of the OCH1 gene is achieved by integration of the ER-retained mannosidase-I from T. reesei, resulting in the expression of a catalytically inactive Och1p (Met1-Ala155° and inadvertently followed by the production of an N-term truncated catalytically active Och1p (OCH1_GSMan5° (FIG. 1°. GlycoSwitchM5 strain shows a similar doubling time as the WT strain, but still lowered cell density at the stationary phase (Jacobs et al., Nat Protoc. 4 (2009° 58-70; Contreras et al., EP1294910B1°. A mutant with improved stability of the GlycoSwitchM5 strain was generated by deleting the upstream copy of the engineered locus, while leaving in the downstream copy with the serendipitous N-terminal alteration (FIG. 1, SuperMan5° (Gehlsen and Chappell, EP2912162B1—Research Corp Technologies, Inc.°. This mutant has an increased genomic stability and produces homogeneous N-glycans for generations after rounds of freezing and thawing and after subsequent transformations.

### Example 1.CRISPR/Cas Editing of the OCH1 Gene in the Wild-Type Pichia Strain NRRL Y-14430

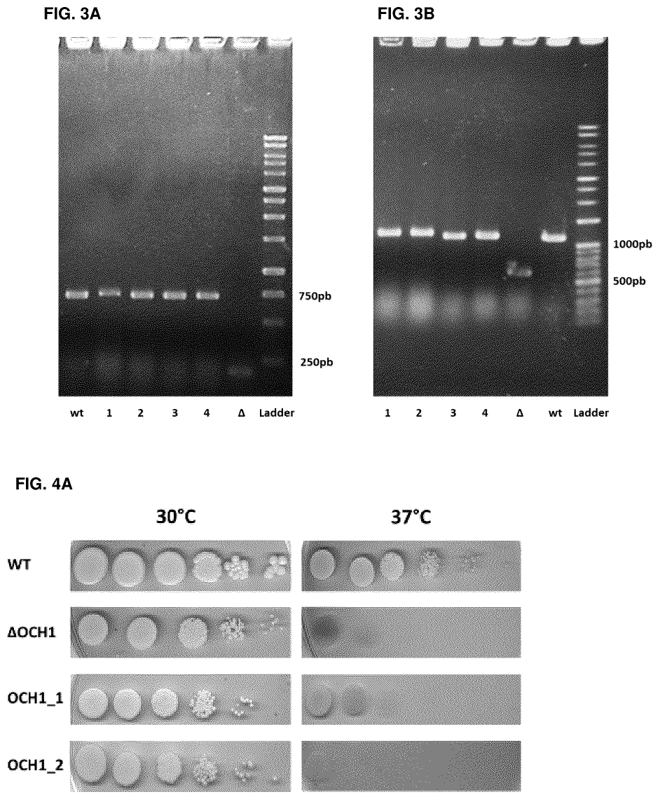

To generate novel Pichia pastoris OCH1 mutants, the CRISPR/Cas9 cassette as present in the pPpT4_pHTXhsCas9 plasmid was used (FIG. 2A° (obtained from Weninger, et al., J Biotechnol, 2016. 235: p. 139-149°. The guideRNA insertion (SEQ ID NO:10° on the pPpT4_pHTXhsCas9 plasmid was confirmed by amplification of a 500 bp fragment (FIG. 2B°. Restriction analysis confirmed the plasmid integrity as shown in FIG. 2C by the presence of a band around 8 kb. After transformation of Pichia pastoris NRRL Y-11430 with pPpT4_pHTXhsCas9-OCH1-gRNA3, the clone screening performed by PCR and sequencing led to the identification of several mutants. One of them consisted of a big deletion evidenced by the amplification of fragments smaller than expected ones (approximately 715pb and 1150 bp for primers 5-6 (SEQ ID NO:5-6° and 3-4 (SEQ ID NO:3-4°, respectively, called ΔOCH1 or OCH1_full KO° (FIG. 3°. ΔOCH1 has a large deletion of 537 bp, which starts 83 bp upstream of the OCH1 coding sequence and truncates the first 151 amino acids of Och1p, which completely abolishes the expression of the protein (FIG. 1, OCH1_full KO°. The other mutants presented small deletions that were identified by sequencing: two of them led to small changes in the protein (OCH1_1 and OCH1_2°. The mutations of the OCH1_1 clone (called OCH1_E151del) were located near to the active site (positions 183-185° and involved a deletion of a negatively charged glutamic acid (E151° amino acid that. For the OCH1_2 mutant, a deletion of amino acids 150P, 151E and 152V, was identified, which are located around the same position of the mutation obtained in the OCH1_1 clone.

Characterization was initially performed by checking their growth rate, temperature sensibility and glycan profiles. In terms of cell growth, it was observed that mutant strains presented a slow growth in comparison to the wild type strain. Moreover, it was observed an increase in temperature sensibility in the mutant strains, which was rescued by addition of 1M Sorbitol to the medium (FIG. 4°. Although all the obtained OCH1 mutants displayed the above-mentioned phenotypic characteristics, the severity of the growth delay and temperature sensibility seemed to correlate with the specific mutation. In this sense, the mutant with the biggest deletion (ΔOCH1° presented the most impaired phenotype, while the single amino acid deletion (OCH1_1 or OCH1_E151del° showed an attenuated growth phenotype, thus being less severely impaired (FIG. 4°. Based on the phenotype of OCH1_E151del, the single amino acid deletion seems to be important for the enzyme activity. A similar situation was observed for OCH1_2 mutant, which showed a deletion of amino acids 150P, 151E and 152V, which are located around the same position of the mutation obtained in the OCH1_1 clone. However, it is evident that OCH1_2 showed a more severe temperature sensitivity, and a lower recovery after sorbitol treatment, than that observed for OCH1_1 mutant, showing the importance for enzyme activity of the deleted three amino acids (FIG. 4°.

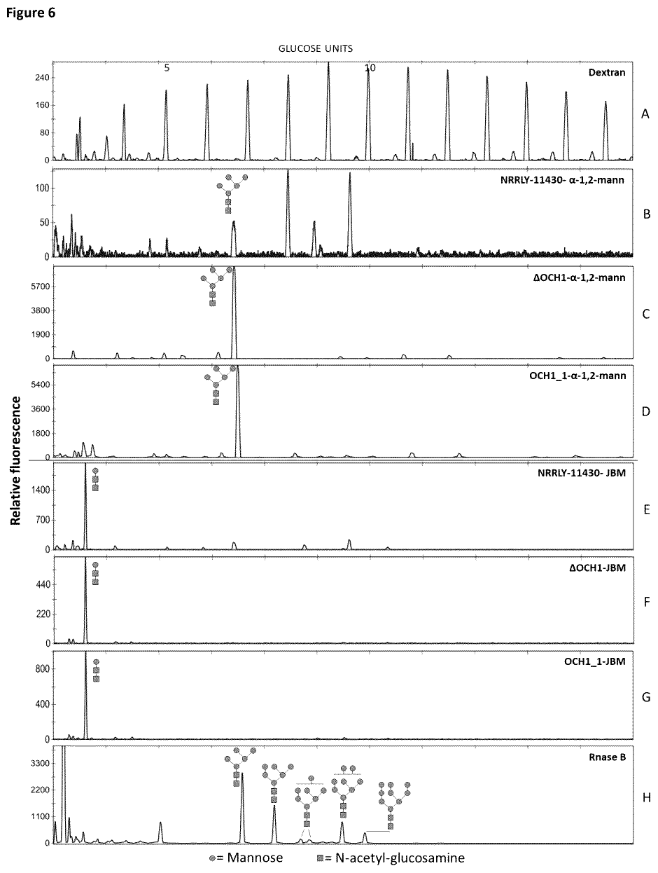

Glycan analysis showed differences in the N-glycosylation pattern between the P. pastoris NRRL Y-11430 WT and the mutated strains. In the former, the main peak corresponded to mannose 9 (M₉° glycoforms, with small amounts of M₈glycoforms. In contrast, OCH1 mutants showed a shift from M₉ to M₈ as the main glycoform (FIG. 5°. Further glycan characterization was performed by N glycan digestion using the enzymes α-1-2-mannosidase and α-1-2,3,6 mannosidase (Jack Bean Mannosidase, JBM°. As observed in FIG. 6, the N-glycans observed in the mutant strains were reduced to M₅ after the treatment with α-1-2-mannosidase, while JBM treatment led to a unique peak corresponding to the basic N-glycan structure (GlcNAc-GlcNAc-Man° (FIG. 6° In contrast, after treatment with α-1-2-mannosidase, the high mannose N-glycans from the P. pastoris NRRL Y-11430 WT strain were reduced to M₉, while the treatment with JBM led to unique peak similar that observed for the mutant strains (FIG. 6°.

Two mutants were selected for further analysis and recombinant protein expression, based on their temperature sensitivity, cell morphology and glycosylation profile: OCH1_1 (OCH1_E151del) and ΔOCH1 (OCH1_full KO) (FIG. 1°. The genotypes of the two knock-out strains were confirmed by ILLUMINA sequencing and no other mutations were found that could be causative of the observed phenotype.

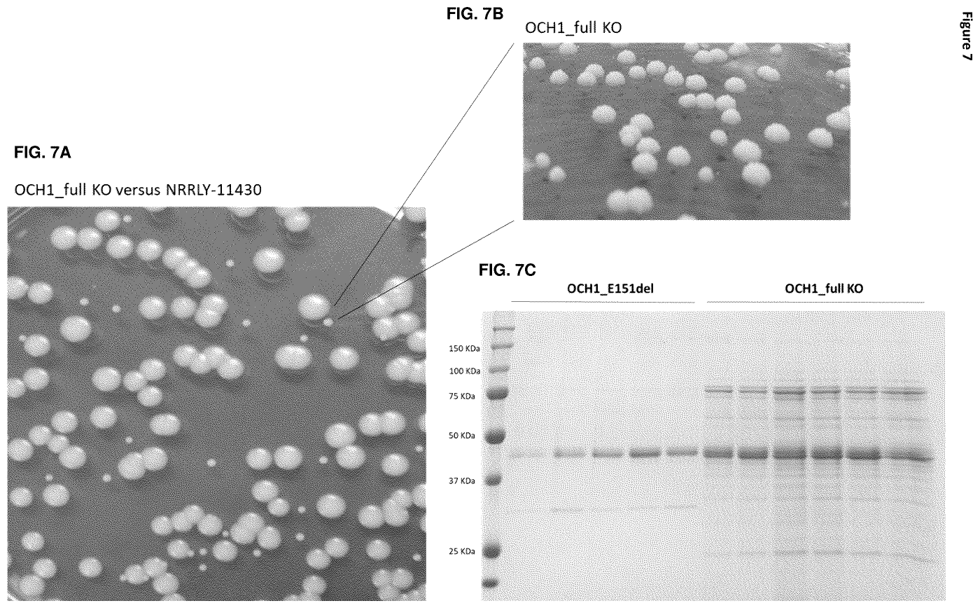

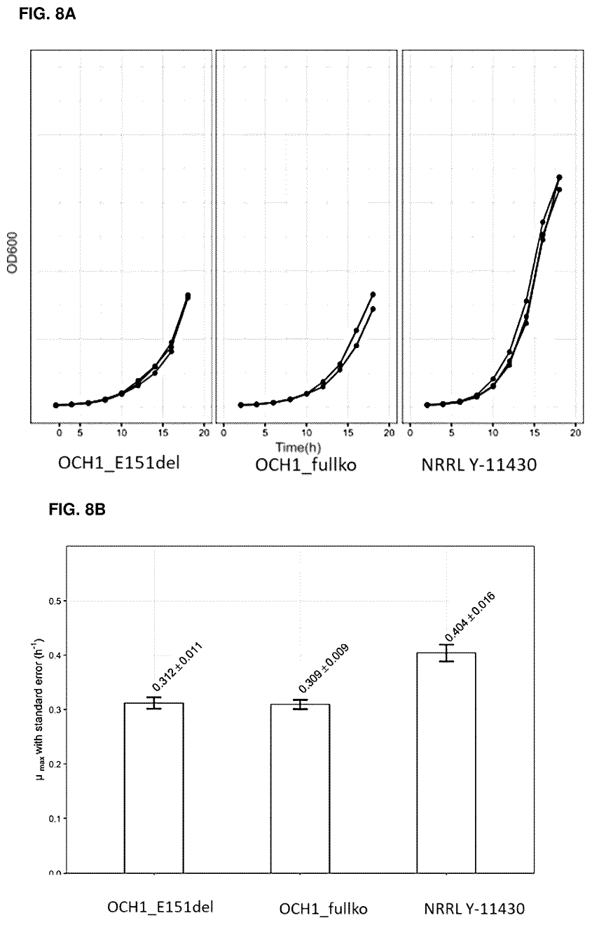

Interestingly, when looked at in more detail, these two mutants show different morphological characteristics. The OCH1_E151del has a smooth colony morphology, almost indistinguishable from the wild type strain, whereas the complete deletion of OCH1 has a wrinkled colony morphology (FIG. 7, 12°. Both strains have a slower specific growth rate in shake flasks compared to the parental wild type strain NRRL Y-11430 though (FIG. 8°.

### Example 2. Recombinant Expression of an Antibody in the Pichia pastoris OCH1 Mutant Strains

Antibodies are emerging as effective anti-viral therapies against highly virulent virus infections, but current manufacturing technologies are extremely complex, time consuming and require high investments. The methylotrophic yeast Komagataella phaffii (syn. Pichia pastoris° is one of the preferred hosts for production of biopharmaceuticals One hurdle precluding Pichia for the manufacturing of Fc-containing drugs being that the yeast glycosylation pathway produces glycoproteins modified with yeast-specific high-mannose oligosaccharide glycans rather than mammalian complex glycans, which may be immunogenic and lead to rapid clearance. Several glycoengineered strains able to produce human-type glycosylation have been reported in the literature, as also provided herein. Alternatively, the glycosylation site can be removed, which results in an antibody deprived of an active effector function (Drug Approval Package: VYEPTI, (n.d.°. https://www.accessdata.fda.gov/drugsatfda_docs/nda/2020/7611190rig1s000TOC.cfm (accessed Sep. 2, 2021°. The second hurdle is that the protein secretion machinery for IgG is less efficient than in mammalian cells. One of the reasons that make antibody manufacturing complex is the fact that they are hetero-multimeric proteins, and two genes need to be transcribed, translated, and folded in a quaternary structure to have a functional molecule. Heavy-chain antibodies are made by two identical single chains that are expressed from a single gene, therefore the recombinant expression of heavy-chain antibodies is easier. Recently, the fusion construct between VHHs and human Fc has been used to increase the avidity and prolong its half-life. These fusion proteins lack the CH1 domain of antibodies, which is notoriously known to be the slowly folding domain of antibodies, therefore maintaining the binding capacity, effector functionality, and prolonged half-life in a single gene format (Feige, et al. Mol. Cell. 34 (2009° 569-579; Godakova, et al. Toxins (Basel°. 11 (2019° 464; Schepens et al., Sci. Transl. Med. 13, eabi7826 (2021° 24°. To address both hurdles, we used the two selected OCH1 mutant strains to transform with the expression plasmid for an antibody VHH-Fc construct herein.

Influenza A viruses are respiratory viruses with a high mutation rate that circulate in many warm-blooded species and are therefore considered a major public health threat of our time. The ectodomain of the Matrix protein 2 (M2e° is highly conserved among the different influenza A subtypes, hence it is considered a good target for a broadly protective vaccine or therapeutic (Saelens, The Journal of Infectious Diseases. 219 (2019° S68-S74°. Previously generated influenza M2e-specific VHHs were used as a VHH-Fc protein for antibody production in glyco-engineered Pichia pastoris OCH1 mutants, under control of the AOX1 promoter and fused to the Ost1 secretion signal. Higher levels of host cell proteins were observed in the medium during recombinant protein expression in the OCH1_fullKO strain as compared to the OCH1_E151del, likely due to an increased cell stress and consequently cell lysis (FIG. 7C°.

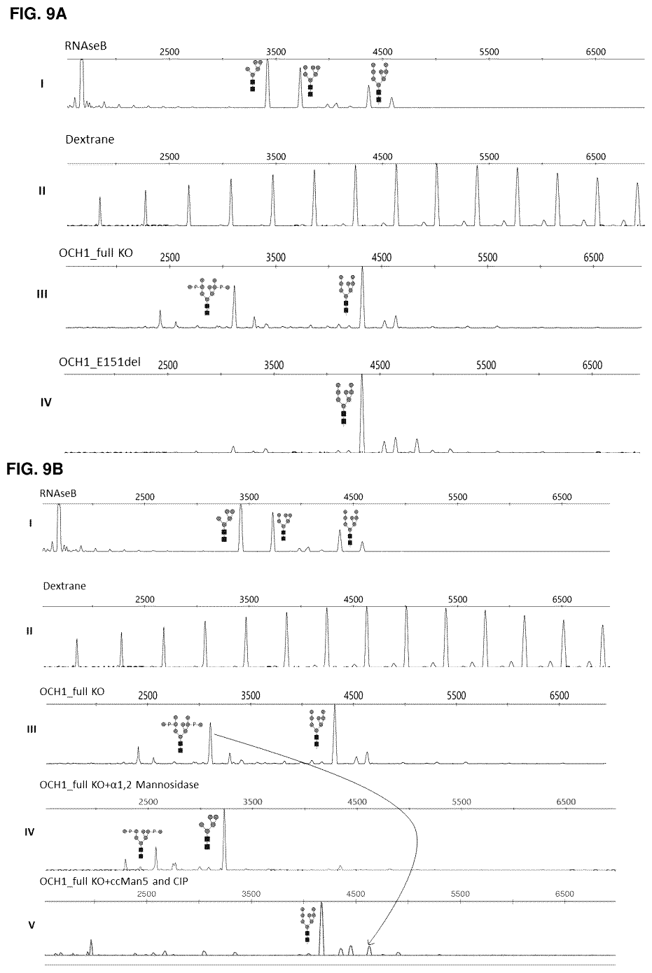

The glycosylation profile of secreted M2eVHH-Fc was analyzed by capillary electrophoresis and both mutants showed the typical OCH1 knock-out glycosylation profile with the majority of the glycans being Man₈GlcNAc₂ (FIG. 9A°. However, smaller peaks appeared at the right of the main peak, which are probably the results of activity of other Golgi-resident mannosyltransferases on the unnatural Man₈GlcNAc₂ substrate. In addition, in the OCH1_fullKO some high-intensity peaks at the left of the main Man₈ peak were observed (FIG. 9A°. After digestion with α1-2 mannosidase from Cellulosimicrobium cellulans (CcMan5° and calf intestinal alkaline phosphatase (CIP°, these peaks moved to the right of the Man₈GlcNAc₂, which identify them as phosphorylated glycans that migrate faster than non-charged glycans because of the extra negative charges of the phosphate groups (FIG. 9B°. As the phosphomannosyltransferases are upregulated in cell-wall stress conditions [30], the higher levels of phosphorylation produced by the OCH1_fullKO strain confirm that this mutant has a cell wall integrity defect. Apart from the phosphorylated glycans, the glycosylation profile of the two OCH1 mutants is identical (FIG. 9A panel IV and FIG. 9B panel V°, which means that a full knock-out of the OCH1 gene is not necessary to obtain the desired glycosylation profile and the OCH1_E151del mutant must be catalytically inactive, with no or very little no residual α1,6-mannosyltransferase activity.

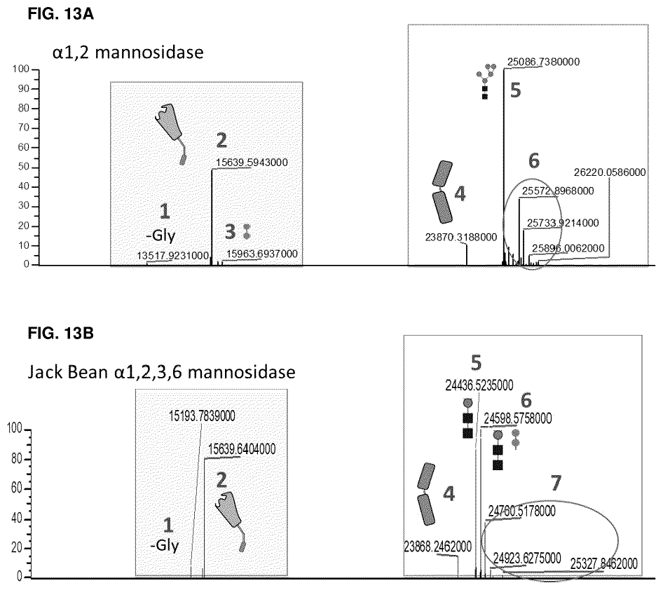

After purification, the M2eVHH-Fc protein was analyzed on LC-MS after digestion with FabRICATOR enzyme (Genovis»FabRICATOR Enzyme, (n.d.°. https://www.genovis.com/products/igg-proteases/fabricator/fabricator-enzyme/(accessed Nov. 4, 2021°°, which cleaves in the CH2 domain just below the hinge region. This results in two protein fragments upon TCEP and iodoacetic acid reduction of the hinge S—S bond, the smaller at 15,642.53 Da corresponding to the VHH+hIgG hinge and the bigger one of 23,872.05 Da corresponding to the CH2 and CH3 domains of the human IgG1 Fc (calculated as monoisotopic masses on Expasy°. The Fc N-glycosylation site is occupied in >95% of the M2eVHH-Fc and it carries mainly Man8GlcNAc2 glycan, with a minority of higher mannose species, confirming what was detected in capillary electrophoresis of the N-glycans of M2eVHH-Fc containing culture medium (FIG. 9°. To investigate the nature of the protein-linked glycans, we incubated the purified protein with α1,2 mannosidase and with α1,2/3/6 jack bean mannosidase before analysis. Overnight treatment with α1,2 mannosidase resulted in a main peak at Man5GlcNAc2 (FIG. 13° and smaller peaks at higher molecular mass (FIG. 13°, consistent to what was observed on capillary electrophoresis and the prior knowledge that Pichia OCH1 knock-out glycoprofile contains glycans modified by other α1,6 mannosyltransferases in the Pichia Golgi (FIG. 9°. Digestion with α1,2/3/6 jack bean mannosidase could remove the O-mannose modifications on the VHH-hinge demonstrating that no β-mannose was present (FIG. 13B°. The O-glycosylation in the VHH-hinge part was reduced and only species with 2 hexoses were observed.

Binding of purified M2eVHH-Fc to different M2e peptide variants was evaluated by enzyme-linked immunosorbent assay (ELISA°. The peptides of the M2e from influenza A/consensus human (H3N2°, A/swine/Belgium/1998 (H1N1° and A/swine/Ontario/2001 (H3N1° were investigated (SEQ ID NO:12-14°. As expected, similarly to the monovalent M2eVHH (M2e-VHH-23m (De Vlieger, et al., (2019° Front. Immunol. Vol10, Art2920°, the VHH-Fc fusion can bind to the human consensus M2 peptide (data not shown°, expressed by the majority of human H3N2 strains, but not to the currently circulating swine influenza viruses, as the M2eVHH epitope extends beyond the universally conserved N-term 8 amino acids of M2e.

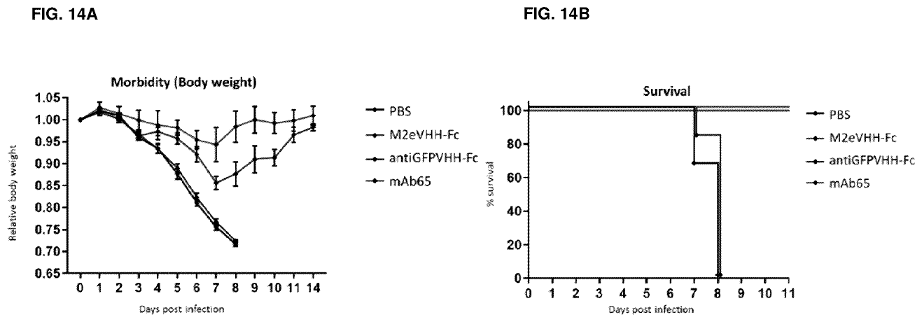

In a proof of concept experiment, the Pichia-produced M2eVHH-Fc produced in the OCH1-E151del strain was tested for efficacy in protecting mice in an influenza challenge model. BALB/c mice were administrated intranasally with 20 μg of M2eVHH-Fc. PBS excipient and antiGFP-Fc served as negative controls, and M2e specific mouse IgG2a mAb65 as positive control. Four hours after administration of the antibodies, mice were infected with 2× LD50 of A/X47 H3N2 influenza virus. Body weight was monitored for 14 days. Mice that received M2eVHH-Fc and mAb65 showed a maximum decrease in body weight of 15% and 5% at day 7 respectively, and all of the mice that received antibody treatment survived (FIG. 14°. The Pichia-produced M2eVHH-Fc showed to be protective, even if to a lesser extent than mAb65. The difference in potency may be attributed to the difference in affinity for M2e, lower affinity of the human IgG Fc in the M2eVHH-Fc to mice FcγRs and/or faster clearance due to the high-mannose glycosylation of the Pichia-produced molecule. Of note, VHH-Fc molecules are about half of the size of a full-length antibody, therefore about twice the number of molecules were administrated for M2eVHH-Fc as compared to mAbs65. The results sufficed to validate in vivo performance of the yeast-made VHH-Fc format and we moved on with the main objective of this thesis, i.e., setting up and exploring technologies to enhance VHH-Fc manufacturing in Pichia.

Pichia pastoris OCH1_E151del can produce correctly folded M2eVHH-Fc that protects mice challenged with influenza A virus upon intranasal administration. In conclusion, the Pichia pastoris NRRL Y-14430 OCH1_E151del mutant strain shows low cell wall stress similarly as observed in the previously used GlycoSwitchM8 strains, and can thus be applied as an alternative strain for recombinant production of therapeutic antibodies or proteins for which a human-like glycan profile is desired.

### Example 3. Recombinant Production of GAlNS in Pichia pastoris OCH1_151Del Mutant

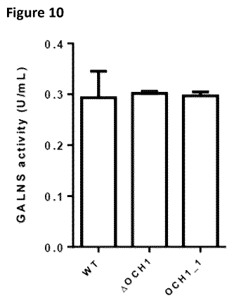

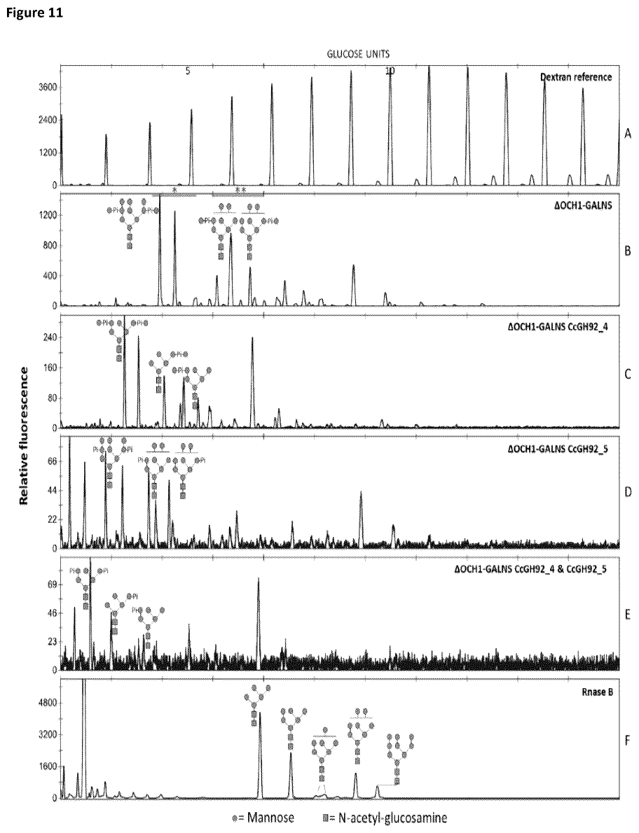

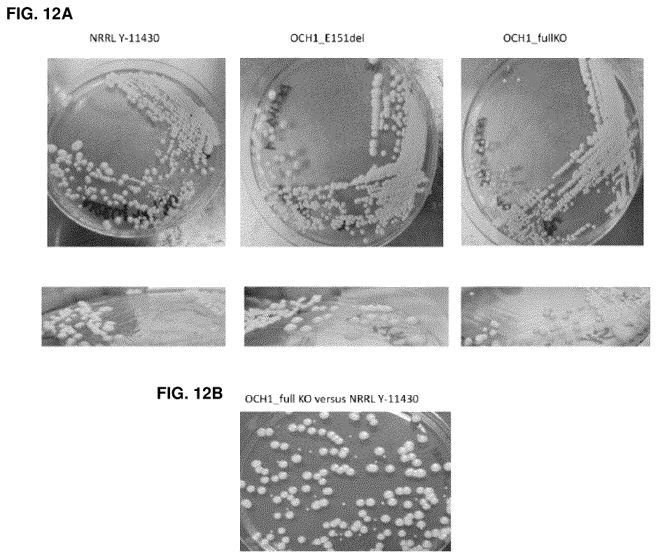

The N-acetylgalactosamine-6-sufate sulfatase (GALNS° is a lysosomal glycoprotein in charge of hydrolyzing the sulfate ester bonds of keratan sulfate and chondroitin-6-sulfate. The human GALNS is composed of 522 amino acids and presents two N-glycosylation sites (N178 and N397° (Mosquera, A., et al., Process Biochemistry, 2012. 47(12°: p. 2097-2102°. Previous reports have suggested that this enzyme is synthetized as a precursor, that is later matured to 40 and 15 kDa peptides linked through disulfide bonds to form 60 kDa monomers and 120 kDa homodimers (Bielicki, and Hopwood, Biochem J, 1991. 279 (Pt 2°: p. 515-20; Masue, et al., J Biochem, 1991. 110(6°: p. 965-70°. In humans, GALNS deficiency causes Morquio A syndrome or Mucopolysaccharidosis type IVA (OMIM #253000°, a human disease characterized by a severe chondrodysplasia, cardiac valve failure, pulmonary compromise, hearing and vision loss, and coarse facial features (Montano, et al., J. Inherit Metab Dis, 2007. 30(2°: p. 165-74°. Recombinant GALNS has been the main therapeutic approach for this disease (Hendriksz, et al., J Inherit Metab Dis, 2014. 37(6°: p. 979-90°, and currently there is enzyme replacement therapy (ERT° available enzyme produced in CHO cells (elosulfase alpha°, which was approved in 2014 for human use (Sanford, and Lo, Drugs, 2014. 74(6°: p. 713-8°. Though, it has been observed that recombinant proteins produced in CHO cells present a highly heterogeneous N-glycosylation pattern that differs from the observed in the native protein and may limit their therapeutic effect (Jin, et al., J Biol Chem, 2018. 293(15°: p. 5572-5584; Wang, et al., J Biosci Bioeng, 2015. 119(6°:p. 657-60°. Human GALNS enzyme has been produced in microorganisms like E. coli and Pichia pastoris, obtaining an active enzyme in both systems, but showing the importance of N-glycosylations on protein stability and cellular uptake (Rodriguez, et al., J Ind Microbiol Biotechnol, 2010. 37(11°: p. 1193-201; Reyes, et al. Sci Rep, 2017. 7(1°: p. 5844°. Although promising findings towards the use of a recombinant GALNS produced in P. pastoris for a Morquio A ERT were reported, this enzyme showed heterogeneous glycoforms with a main glycan type corresponding to 9 mannose residues (M₉°, which is higher than the glycoforms present in the enzyme produced in CHO cells (BisP-Man₇ and BisP-Man₆° (EMA: Assessment report. Vimizin® (elosulfase alfa): http://www.ema.europa.eu/docs/en_GB/document_library/EPAR-_Public_assessment_report/human/002779/WC500169242.pdf.°. Since, in general, N-glycosylation changes respect to the wild-type human protein have been associated with adverse effects (Dicker, and Strasser, Expert Opin Biol Ther, 2015. 15(10°: p. 1501-16°, it is necessary to improve the P. pastoris N-glycosylation pattern, to obtain a more suitable protein for therapeutic purposes and preserving easy handling and scaling up. So, for this purpose, we transformed the OCH1 mutant strains described herein. To express recombinant human GALNS the AGZ-H expression vector was used to transform P. pastoris NRRL Y-11430 ΔOCH1 and OCH1_1 mutant strains. The vector was constructed using modular cloning. The presence of GALNS gene on the generated constructs was confirmed by PCR amplification and restriction analysis and the expression was confirmed for the transformed strains. No significant differences in GALNS activity were observed among the OCH1 mutants and the wild type strains (FIG. 10°. All clones displayed an enzymatic activity of around 0.3 U/mL. these results suggest that the N-glycosylations are not relevant for the activity of the enzyme.

Detailed analysis of purified hprGALNS N-glycosylation profile of P. pastoris NRRL Y-11430 OCH1 mutants (FIG. 5B° showed mainly the presence of M₈glycans similar to the N-glycoforms found in proteins produced in mammalian cells (Clerc, Fet al., Glycoconj J, 2016. 33(3°: p. 309-43; Lalonde, and Durocher, J Biotechnol, 2017. 251: p. 128-140 °.

Overall, these results suggest that OCH1 deficient Pichia pastoris strains offer an easy handling and suitable platforms to produce human-like recombinant lysosomal enzymes.

### Material and Methods.

### Generation of OCH1 Mutants.

The wild-type P. pastoris (syn. Komagataella phaffi° NRRL Y-11430 strain was genetically modified to obtain OCH1 mutants using CRISPR/Cas9 following the protocol described by Weninger, A., et al. (J Biotechnol, 2016. 235: p. 139-149°. For this purpose, the pPpT4_pHTX plasmid, containing the OCH1 gRNA, was used to transform the P. pastoris NRRL Y-11430 strain. Transformant yeast were selected using YPD medium (yeast extract 1% p/v; peptone 2% p/v; dextrose 2%° supplemented with zeocin 100 μg/mL (R25001°. Mutant clones were screened by PCR using two independent set of primers (Primers SEQ ID NOs: 3-6° and sequencing.

Removal of CRISPR/Cas9 Plasmids from P. pastoris OCH1 Mutants.

After confirmation, CRISPR/Cas9 plasmids were removed (cured° from OCH1 mutants by streakout in YPD plates without zeocin. After 48 h incubation at 30° C., colonies from each mutant were selected and cultured in YPD medium during 48 h at 28° C., and restreaked in YPD agar plates without zeocin. To confirm plasmid curing, colonies from the YPD-plate were restreaked on an YPD agar plate supplemented with zeocin 100 μg/mL. After this propagation, the OCH1 mutants were able to grow on YPD media, but did not grow on media supplemented with zeocin.

### Strains and Media.

Escherichia coli (E. coli° MC1061 or DH5a were used for standard molecular biology manipulations. E. coli cultures were grown in LB broth (0.5% yeast extract, 1% tryptone, and 0.5% NaCl with or without 2% agar° at 37° C. Liquid cultures are grown under continuous agitation at 250 rpm in a shaker-incubator. Strains were selected on 50 μg/mL of the appropriate antibiotics: carbenicillin (Duchefa Biochemie°, kanamycin (Sigma Aldrich°, Zeocin® (Life Technologies°, hygromycin (Duchefa Biochemie° or nourseothricin (Werner BioAgents°.

Yeast cultures were grown in YPD (1% yeast extract, 2% peptone, 2% D-glucose with or without 2% agar° and selected with 100 μg/ml Zeocin®, or 100 μg/ml Zeocin® and 500 μg/ml G418 (InvivoGen° at 28° C. in a shaker incubator (250 rpm° or static at 30° C. Transformants were selected using the appropriate combination of antibiotics: 100 μg/mL Blasticidine S—HCl (Sigma-Aldrich°, 100 μg/mL Zeocin®, 500 μg/mL Geneticin/G418 (Life Technologies°, 300 μg/mL Hygromycin B and 100 μg/mL Nourseothricin.

### Phenotypic Characterization of OCH1 Mutants.

Phenotype of OCH1 mutants was characterized by colony morphology and temperature sensibility in YPD plates. Colony morphology was evaluated at macroscopic and 4× stereoscopic magnification. In addition, microscopic evaluation was performed by direct observation (100× magnification° of samples taken from liquid cultures in YPD medium at 10 mL scale. Temperature sensibility was determined as the ability to growth at 30 and 37° C. after 72 h incubation. In addition, to further characterize the cell-wall defect on OCH1 mutants, a rescue assay was performed by culture in YPD plates supplemented with 1M sorbitol (Popolo, et al., Med Mycol, 2001. 39 Suppl 1: p. 111-21°.

### Plasmid Construction of the M2eVHH-Fc Expression Vector by Golden Gate Based Modular Cloning.

The vector was generated using an adapted version of the Yeast Modular Cloning toolkit based on Golden Gate assembly (Vanluchene, dissertation, Ghent University, 2020. http://hdl.handle.net/1854/LU-8684279; Lee, et al., ACS Synth. Biol. 4 (2015° 975-986°. The coding sequence of the secretion signal was designed as part 3a, the VHH as part 3b, the human IgG1 hinge-human IgG1 Fc as part 4a. All the sequences were codon optimized for expression in P. pastoris using the IDT proprietary algorithm and ordered as gBlocks at IDT (Integrated DNA Technologies BVBA, Leuven, Belgium°. Each coding sequence was flanked by unique part-specific upstream and downstream Bsal-generated overhangs. The gblocks were inserted in a universal entry vector via BsmBI assembly which resulted in different “part” plasmids, containing a chloramphenicol resistance cassette. Part plasmids were assembled into expression plasmids via a Golden Gate Bsal assembly. The expression plasmid consisted of the assembly of 8 parts: Part_1 left assembly connector, Part_2 promoter, Part_3 coding sequence (possible to split in Part3a and 3b°, Part_4 (possible to split in Part4a and 4b° terminator, Part_5 right assembly connector, Part_6 and Part_7 miscellaneous (usually Pichia antibiotic cassette°, and Part_8 E. coli marker and replication origin. The entry vector and the Part_8 vector contain an RFP or GFP dropout cassette for red or green/white screening. Every part is used in an equimolar amount (20 fmol° and mixed with T4 DNA ligase and BsmBI or Bsal enzyme in a 20 μl reaction in 1× T4 DNA ligase buffer. 25-50 cycles of digestion (42° C. for 2 minutes° and ligation (16° C. for 5 minutes° are followed by a final digestion step (60° C. for 10 minutes°. The assembly reaction is transformed in E. coli and selection of correctly assembled expression plasmids is made in LB supplemented with antibiotic. All the parts and expression plasmids were sequence verified.

Pichia pastoris Transformation

For transformation with expression plasmid, Pichia pastoris strains were transformed according to the lithium-acetate-DTT transformation protocol described by Wu and Letchworth (BioTechniques, 36 (2004° 152-154°. Briefly, Pichia pastoris competent cells were prepared by incubation in 100 mM LiAc, 10 mM DTT, 0.6 M sorbitol and 10 mM Tris-HCl, pH7.5. Competent cells were washed in 1M sorbitol before transformation by electroporation (1.5 kV, 25 ρF, 200Ω° with 1-2 μg of linearized DNA. Transformants were selected in YPD-agar supplemented with antibiotics at 30° C. for at least 48 hours.

Protein Expression and Purification in Pichia pastoris

For small scale expression screening, single colonies of P. pastoris were inoculated in 2 ml BMGY in a 24 deep well block. Cells were grown in a shaking incubator (28° C., 250 rpm° in BMGY (1% yeast extract, 2% peptone, 100 mM KH₂PO₄/K₂HPO₄, 1.34% YNB, 1% glycerol, pH 6° for 48 hours, then protein expression was induced by changing the medium to BMMY (same composition as BMY but with 1% methanol replacing the 1% glycerol∞. Switch to BMMY was done by harvesting the biomass at 1,500 g, 4° C. for 10 minutes and resuspended it in BMMY. Methanol was spiked into the medium every 8-10 hours at 1% final concentration and after 48 hours the medium was collected by centrifugation at 1,500 g, 4° C. for 5 minutes. Protein expression levels were either evaluated on Coomassie-stained SDS-PAGE of crude supernatant. Crude supernatant was either used immediately for analytics purposes or stored at −20° C.

For protein purification, an overnight culture of P. pastoris was diluted in 125 ml of BMGY to 0.1 OD₀₀ in 2-liter baffled shake flasks. After 48 hours, the medium was switched to BMMY to induce protein expression and induction was maintained for another 48 hours by spiking methanol at 1% final concentration every 8-10 hours. The culture medium was collected by centrifugation at 1,500 g, 4° C. for 15 minutes and filtered over a 0.22 μm bottle top filter (Millipore° before loading on a HiTrap MabSelect SuRe 5 ml column (GE Healthcare°, equilibrated with 1.5 M NaCl, 0.5 M EDTA, 50 mM L-histidine, pH 7. The column was eluted with 100 mM L-Arginine, 100 mM Acetate, 125 mM NaCl pH3.5. Collected fractions were neutralized to pH 6.5 with 1 M Tris-HCl pH 9. Elution fractions containing the protein of interest (evaluation on SDS-PAGE° were pooled, injected on a HiLoad 16/600 Superdex 200 μg size-exclusion column (GE-Healthcare° and eluted with 25 mM L-His, 125 mM NaCl, pH 6. After spectroscopic protein concentration determination (absorbance at 280 nm minus buffer blank°, purified protein was concentrated using Amicon 10 kDa MWCO spin columns if required, snap-frozen in liquid nitrogen, and stored at −80° C.

### N-Glycosylation Analysis of M2e-VHH-Fc by Capillary Electrophoresis

N-glycans on secreted proteins were analyzed by DNA sequencer-assisted flurophore-assisted carbohydrate electrophoresis (DSA-FACE° (Laukens, et al., A. Castilho (Ed.°, Glyco-Engineering: Methods and Protocols, Springer, New York, NY, 2015: pp. 103-122; Callewaert, et al. Glycobiology. 11 (2001° 275-281°. At the end of the production phase, cells were harvested by centrifugation and one volume of supernatant is mixed with two volumes of reduction carboxymethylation buffer (8 M urea, 360 mM Tris pH 8.6, 3.2 mM EDTA and samples are denatured at 50° C. for 1 hour. Samples were immobilized on polyvinylidenfluoride (PVDF° on a 96-well multiscreen plate (Millipore° and reduced with DTT. Free thiol groups are blocked with iodoacetic acid and the membrane with 1% polyvinylpyrrolidone. The N-glycans are released by PNGaseF in 10 mM tris-acetate at 37° C. (in-house produced. The released glycans were labeled overnight at 37° C. by reductive amidation with 10 mM aminopyrene trisulfonate (APTS° in citric acid and 1 M NaCNBH₃ in Dimethyl sulfoxide (DMSO°. The excess of APTS was removed by size exclusion chromatography over a Sephadex G10 (GE Healthcare° resin, and labeled glycans are analyzed on an ABI 3130 capillary DNA sequencer (Applied Biosystems°. N-glycans of bovine pancreas RNase B (Man5-9GlcNAc2° and a dextran ladder consisting of α-1,6-linked glucose residues (Glucose Units° were both included as references. Data was analyzed with the Genemapper software (Applied Biosystems°. Exoglycosidase treatment was performed on labeled glycans with Hypocreajecorina α-1,2-mannosidase (produced in our laboratory, 0.33 μg per digest° α-1,2/3/6 Jack Bean mannosidase (in-house produced, 19 mU per digest° and Streptococcus pneumoniae β-NAcetylhexosaminidase (Prozyme, 4 mU per digest° overnight at 37° C. in 20 mM sodium acetate (pH 5.2 °.

### In Vitro Binding by ELISA

Microtiter plates (F96 Maxisorp, Nunc° were coated overnight at 37° C. with 100 ng of M2e peptides (SEQ ID Nos:12-14°. After washing, with PBS supplemented with 0.05% Tween-20 (PBST°, the plates were blocked for 1 h with 5% milk in PBS after which they were washed again 3 times with PBS and once with PBST. Serial dilutions of the VHH-Fc were added and the bound proteins were detected mouse anti-His antibody (1:3000° and anti-mouse-HRP (1:5000°. After washing with PBST, 50 μl of TMB substrate (Tetramethylbenzidine, BD OptETA° was added to every well. The reaction was stopped by addition of 50 μl of 1M H2SO4, after which the absorbance at 450 nM was measured with an iMark Microplate Absorbance Reader (Bio Rad°. The EC50 value was estimated using the Binding-Saturation ‘one-site total’ binding equation in GraphPad Prism.

### Challenge Experiment in Mice

Female BALB/c mice were purchased from Charles River (Charles River Wiga, Sulzfeld, Germany°. The animals were housed under specific pathogen-free conditions and in a temperature-controlled environment with 12 h light/dark cycles; food and water were provided ad libitum. The animal facility operates under the Flemish Government License Number LA1400536. All experiments were authorized by the Institutional Ethical Committee on Experimental Animals (Ethical application EC2018-012°. For intranasal treatment and infection mice were anesthetized with isoflurane. Four hours after intranasal administration of 20 μg VHH23-Fc, GBP-Fc (Fc fusion of the GBP control nanobody°, 20 μg of the M2e specific mouse IgG2a monoclonal antibody (positive control° or PBS mice were infected with 2×LD50 of A/X47 (H3N2° influenza virus. Body weight loss was monitored for 14 days. Mice that lost more than 25% of their initial body weight were sacrificed by cervical dislocation. Differences in body weight loss were analyzed by 2-way ANOVA. Differences in survival were tested by a Log-rank (Mantel-Cox° test. Statistical analysis was performed using GraphPad Prism 9.1.

### Protein Analytical Techniques

Proteins were analyzed on 12% Tris-Glycine SDS-PAGE gels or GenScript ExpressPlus™ PAGE Gel, 4-20%. Samples were mixed with Laemmli loading dye (200 mM Tris-HCl, pH 6.8, 40% glycerol, 10% SDS, 0.8% bromophenol blue with or without 30 mM DTT° and heat denatured at 98° C. for 10 minutes. For visualization the gels were stained with Coomassie (0.1% Coomassie, 40% methanol, 10% acetic acid° for 1 hours and destained (40% methanol, 10% acetic acid° for 4-6 hours.

When needed, samples were precipitated prior analysis using the DOC-TCA protocol. Briefly, 1 ml of culture supernatant was mixed with 100 μl Na-deoxycholic acid (DOC, 5 mg/mL° and 110 μL saturated (w/v° trichloroacetic acid (TCA°. The samples were centrifuged at maximum speed and the pellet is washed with acetone followed by 70% ethanol and resuspended in PBS after being dried. The protein content of the lysates was determined using BCA protein assay kit (Pierce°.

Protein deglycosylation using PNGaseF (New England Biolabs° was done following the NEB-protocol. DOC-TCA purified proteins were denatured in glycoprotein denaturation buffer (0.5% SDS, 40 mM DTT° for 5 minutes at 98° C., then supplemented with 1% NP-40 and 50 mM Sodium Phosphate pH 7.5 and 7.6 IUM mU PNGaseF (produced in house°. Samples were analyzed on SDS-PAGE after overnight incubation at 37° C.

For western blot of GALNS samples, crude protein extracts from 100 mL cultures were analysed by SDS polyacrylamide gel electrophoresis (PAGE° under reducing conditions. Samples were electrotransferred to a nitrocellulose membrane (Hybond C-Extra; Amersham Bioscience, Piscataway, NJ, USA°. The recombinant GALNS was recognized with an α-His antibody (6× His Tag Antibody Dylight™,800 Conjugated. Rockland 200-345-382°.

For intact protein mass spectrometry, purified M2eVHH-Fc protein (10 μg° was pre-treated with 10 U of FabRICATOR enzyme (Genovis° at 37° C. for 2 h and then reduced with tris(2-carboxyethyl°phosphine (TCEP, 10 mM° for 30 min at 37° C. The reduced protein was separated on an Ultimate 3000 HPLC system (Thermo Fisher Scientific, Bremen, Germany° connected to an LTQ Orbitrap XL mass spectrometer (Thermo Fischer Scientific°. Briefly, approximately 8 μg of protein was injected on a Zorbax 300SB-C18 column (5 μm, 300 Å, 1×250 mm IDxL; Agilent Technologies' and separated using a 30 min gradient from 5% to 80% solvent B at a flow rate of 100 μl/min (solvent A: 0.1% formic acid and 0.05% trifluoroacetic acid in water; solvent B: 0.1% formic acid and 0.05% trifluoroacetic acid in acetonitrile°. The column temperature was maintained at 60° C. Eluting proteins were directly sprayed in the mass spectrometer with an ESI source using the following parameters: spray voltage of 4.2 kV, surface-induced dissociation of 30V, capillary temperature of 325° C., capillary voltage of 35V and a sheath gas flow rate of 7 (arbitrary units°. The mass spectrometer was operated in MS1 mode using the orbitrap analyzer at a resolution of 100,000 (at m/z 400° and a mass range of 600-4000 m/z, in profile mode. The resulting MS spectra were deconvoluted with the BioPharma FinderTM3.0 software (Thermo Fischer Scientific° using the Xtract deconvolution algorithm (isotopically resolved spectra. The deconvoluted spectra were manually annotated.

### Human Recombinant GALNS Production

The sequence used for GALNS expression was codon-optimized for Pichia pastoris as previously described (Espejo-Mojica, et al., Universitas Scientiarum, 2016. 21: p. 195-217). AGZ-H Expression vectors carrying a GALNS cDNA without N-terminal His-tag were transformed into cured OCH1 mutants to express recombinant human GALNS protein (hprGALNS). Transformants were selected using YPD plates supplemented with zeocin 100 μg/mL. Tranformation was confirmed by PCR using GALNS specific primers (Primers 7 and 8; SEQ ID NOs: 7-8), while the expression of hprGALNS was followed up by western blot using an α-His antibody (6× His Tag Antibody DylightT™ 800 Conjugated. Rockland 200-345-382). In addition, expression cassette insertion was confirmed by amplification using primers (Primers 7 and 9; SEQ ID NOs: 7 and 9) for AOX1 gene.

### Cell Growth Characterization

One clone of P. pastoris NRRL Y-11430 and one clone of P. pastoris NRRLY-11430-ΔOCH1 were grown by triplicate in 60 mL of YPD medium until reaching 1.2 O.D. at 28° C. and 200 RPM. Cells were harvested by centrifugation (4000 RPM 20 min at 4° C.) and the pellet was resuspended in 100 mL of BMGY medium and cultured for 30 h at 30° C. and 200 RPM. Finally, cells were recovered and resuspended in 100 mL of BMMY medium, and cultured for 96 h at 30° C. and 250 RPM. Methanol was added every 24 h to maintain a final concentration of 0.5%. Aliquots were taken every 24 h and stored at −20° C. until their use. The aliquots were centrifuged and GALNS activity was measured in the supernatant.

### Shakeflask Cultures for GALNS Production and Activity Analysis

The P. pastoris Y-11430-ΔOCH1-GALNS were grown at a scale of 100 mL. For this, cells were grown in 100 mL of YPD medium during 24 h at 28° C. and 250 RPM. Cells were harvested by centrifugation (4000 RPM 20 min at 4° C.) and the pellet was resuspended in 100 mL of BMGY medium and cultured for 48 h at 30° C. and 250 RPM. Finally, cells were recovered and resuspended in 100 mL of BMMY medium, and cultured for 96 h at 30° C. and 250 RPM. Methanol was added every 24 h to maintain a final concentration of 0.5%. Aliquots were taken every 24 h and stored at −20° C. until their use. The aliquots were centrifuged and GALNS activity was measured in the supernatant.

### Enzyme Activity

GALNS activity was assayed by using the substrate 4-methylumbelliferyl-β-d-galactopyranoside-6-sulfate (Toronto Chemicals Research, North York, ON, Canada), following a previously reported protocol (van Diggelen, et al., Clin Chim Acta, 1990. 187(2): p. 131-9). Briefly, 10 μL of the sample and 20 μL of 2 mM substrate solution (pH=4,3) were incubated at 37° C. per 17 hours, after which β-galactosidase (from Aspergillus oryzae) (SIGMA ALDRICH G5160-25KU, Japan), was added to the mixture and incubated at 37° C. per 2 hours more. The enzymatic reaction was stopped by adding 0,17M Sodium carbonate buffer pH 9.8 containing 0,24M of glycine. Fluorescence was measured in a Modulus Fluorometer using 360 nm as excitation wavelength and 415 nm as emission wavelength. Fluorescence readings were compared to the measured fluorescence of a standard solution containing 1 nM of 4-methylumbelliferone. One unit (U) was defined as the amount of enzyme catalyzing 1 nmol substrate per hour. Specific GALNS activity was expressed as U mg₋₁ of protein as determined by BCA protein assay kit (Thermo).

### N-Glycan Analysis and Exoglycosidase Digestion of GALNS Proteins

OCH1 mutants were grown in 2 mL of YPD medium in 24 deed well plates and cultured at 28° C. and 200 RPM up to an OD₆₀₀ of 1.5. hprGALNS-producing clones were grown in 2 mL of BMGY medium (yeast extract 1% p/v; peptone 2% p/v; potassium phosphate 100 mm ph 6.0; yeast nitrogen base 1.34%; biotin 4×10₋₅%; glycerol 1%), and cultured for 24 h at 28° C. and 200 RPM. After incubation, the cultures were centrifuged at 2500 RPM 10 minutes at 4° C., the medium was removed and cells were resuspended in BMMY medium (potassium phosphate 100 mM pH 6.0; yeast nitrogen base 1.34%; biotin 4×10₋₅%; methanol 0.5%), and cultured for 24 h at 28° C. and 200 RPM. For the N-glycan analysis the samples were labeled with 8-aminopyrene-1.3.6-trisulphonic acid (APTS° according to a published protocol (Laroy, et al. Nat Protoc, 2006. 1(1°: p. 397-405°. Then, N-linked glycans were prepared by blotting on 96-well plate with PVDF membrane plates (Millipore, Bedford, UK°, and analyzed by capillary electrophoresis with laser-induced fluorescence detection (CE-LIF° using an ABI 3130 DNA sequencer, as previously reported (Laroy et al.° RNAse B and Dextran were used as reference patterns. To further characterize the N-glycan structure, protein samples were digested using α1-2 manossidase, α1-2,3,6 mannosidase (Jack Bean Mannosidase, JBM°, and CcGH92_4 and CcGH92_5 mannosidases from C. cellulans, previously produced by Callewaert group at the VIB-UGENT center for medical biotechnology (Tiels, et al., Nat Biotechnol, 2012. 30(12°: p. 1225-31°. After digestion, samples were analyzed through CE-LIF as described above.

### Statistical Analysis

Differences between groups were tested for statistical significance by using two-way ANOVA and Tukey's multiple comparison test. An error level of 5% (p<0.05) was considered significant. All analyses were performed using GraphPad Prism v.7.0 (GraphPad Software, La Jolla, California, USA). All results are shown as mean±SD.

### REFERENCES

[1] C. P. Kurtzman, Biotechnological strains of Komagataella (Pichia) pastoris are Komagataella phaffii as determined from multigene sequence analysis, J. Ind. Microbiol. Biotechnol. 36 (2009) 1435-1435.
[2] G. H. Wegner, Emerging applications of the methylotrophic yeasts, FEMS Microbiology Reviews. 7 (1990) 279-283.
[3] J. M. Cregg, K. J. Barringer, A. Y. Hessler, K. R. Madden, Pichia pastoris as a host system for transformations, Molecular and Cellular Biology. 5 (1985) 3376-3385.
[4] J. L. Cereghino, J. M. Cregg, Heterologous protein expression in the methylotrophic yeast Pichia pastoris, FEMS Microbiology Reviews. 24 (2000) 45-66.
[5] S. Braun-Galleani, J. A. Dias, A. Y. Coughlan, A. P. Ryan, K. P. Byrne, K. H. Wolfe, Genomic diversity and meiotic recombination among isolates of the biotech yeast Komagataella phaffii (Pichia pastoris), Microbial Cell Factories. 18 (2019) 211.
[6] F. S. Hartner, A. Glieder, Regulation of methanol utilisation pathway genes in yeasts, Microbial Cell Factories. 5 (2006) 39.
[7] J. M. Cregg, K. R. Madden, K. J. Barringer, G. P. Thill, C. A. Stillman, Functional characterization of the two alcohol oxidase genes from the yeast Pichia pastoris, Molecular and Cellular Biology. 9 (1989) 1316-1323.
[8] S. B. Ellis, P. F. Brust, P. J. Koutz, A. F. Waters, M. M. Harpold, T. R. Gingeras, Isolation of alcohol oxidase and two other methanol regulatable genes from the yeast Pichia pastoris, Molecular and Cellular Biology. 5 (1985) 1111-1121.
[9] M. Hasslacher, M. Schall, M. Hayn, R. Bona, K. Rumbold, J. Luckl, H. Griengl, S. D. Kohlwein, H. Schwab, High-level intracellular expression of hydroxynitrile lyase from the tropical rubber tree Hevea brasiliensis in microbial hosts, Protein Expr Purif. 11 (1997) 61-71.
[10] Kalbitor (ecallantide) FDA Approval History, Drugs.Com. (n.d.). https://www.drugs.com/history/kalbitor.html (accessed Sep. 28, 2021).
[11] Pichia Produced Products on the Market I Pichia Pastoris Protein Expression Platform: Eukaryotic Protein Expression, (2012).
[12] S. C. Spohner, H. Müller, H. Quitmann, P. Czermak, Expression of enzymes for the usage in food and feed industry with Pichia pastoris, Journal of Biotechnology. 202 (2015) 118-134.
[13] Meat-free outsells beef, Nature Biotechnology. 37 (2019) 1250-1250.
[14] Aebi, M., N-linked protein glycosylation in the ER. Biochim Biophys Acta, (2013). 1833(II): p. 2430-7.
[15] Delic, M., et al., The secretory pathway: exploring yeast diversity. FEMS Microbiol Rev, (2013). 37(6): p. 872-914.
[16] Laukens, B., C. De Visscher, and N. Callewaert, Engineering yeast for producing human glycoproteins: where are we now? Future Microbiol, (2015). 10(1): p. 21-34.
[17] Choi, B. K., et al., Use of combinatorial genetic libraries to humanize N-linked glycosylation in the yeast Pichia pastoris. Proc Natl Acad Sci USA, (2003). 100(9): p. 5022-7.
[18] Nakayama et al., OCH1 encodes a novel membrane bound mannosyltransferase: outer chain elongation of asparagine-linked oligosaccharides., EMBO Journal. 11 (1992) 2511-2519.
[19] T. Nagasu, Y. Shimma, Y. Nakanishi, J. Kuromitsu, K. Iwama, K. Nakayama, K. Suzuki, Y. Jigami, Isolation of new temperature-sensitive mutants of Saccharomyces cerevisiae deficient in mannose outer chain elongation, Yeast. 8 (1992) 535-547.
[20] F. W. Krainer, C. Gmeiner, L. Neutsch, M. Windwarder, R. Pletzenauer, C. Herwig, F. Altmann, A. Glieder, O. Spadiut, Knockout of an endogenous mannosyltransferase increases the homogeneity of glycoproteins produced in Pichia pastoris, Scientific Reports. 3 (2013) srep03279.
[21] W. Vervecken, V. Kaigorodov, N. Callewaert, S. Geysens, K. De Vusser, R. Contreras, In Vivo Synthesis of Mammalian-Like, Hybrid-Type N-Glycans in Pichia pastoris, Applied and Environmental Microbiology. 70 (2004) 2639-2646.
[22] B. Laukens, C. D. Wachter, N. Callewaert, Engineering the Pichia pastoris N-Glycosylation Pathway Using the GlycoSwitch Technology, in: Glyco-Engineering, Humana Press, New York, NY, 2015: pp. 103-122.
[23] A. Pekarsky, L. Veiter, V. Rajamanickam, C. Herwig, C. Grunwald-Gruber, F. Altmann, O. Spadiut, Production of a recombinant peroxidase in different glyco-engineered Pichia pastoris strains: a morphological and physiological comparison, Microbial Cell Factories. 17 (2018) 183.

1. A yeast strain comprising:, which comprises
a mutant alpha-1,6-mannosyltransferase Och1 gene encoding:
an OCH1 α-1,6-mannosyltransferase mutant protein comprising a deletion of the amino acid at position 151 of SEQ ID NO:1 or at a position corresponding to position 151 of a OCH1 homologue of SEQ ID NO:1, and wherein the OCH1 mutant protein is catalytically inactive.

2. The yeast strain of claim 1, wherein the mutant Och1 gene encodes the OCH1 protein of SEQ ID NO:2, or wherein the mutant Och1 gene encodes a protein having at least 90% identity with SEQ ID NO: 2.

3. The yeast strain of claim 1, wherein the mutant Och1 gene is present on a chromosome.

4. The yeast strain of claim 3, herein the mutant Och1 gene has replaced the wild type Och1 gene at the Och1 locus.

5. The yeast strain of claim 1, wherein said the mutant Och1 gene is maintained located on a plasmid, and wherein the wild type Och1 gene on the chromosome has been disrupted.

6. The yeast strain of any of claims 1 to 5 claim 1, wherein the strain produces substantially homogeneous N-glycans with Man₈GlcNAc₂ being the predominant N-glycan form.

7. The yeast strain of claim 1, wherein the yeast strain is a methylotrophic yeast strain.

8. The yeast strain of claim 7, wherein the yeast strain is a Pichia strain.

9. The yeast strain of claim 1, wherein the yeast strain further comprises a nucleic acid molecule encoding a glycoprotein heterologous to the yeast strain.

10. A method for producing a heterologous glycoprotein deficient in hypermannosylated N-glycans in yeast, the method comprising:
introducing a nucleic acid molecule encoding the heterologous glycoprotein in into cells of the yeast strain of claim 1, or and
cultivating the yeast cells to produce the heterologous glycoprotein.

11. A kit for manufacturing of glycoproteins deficient in hypermannosylated N-glycans in yeast, the kit comprising: a yeast strain of claim 1.

12. (canceled)

13. The yeast strain of claim 1, wherein the yeast strain is Pichia pastoris NRRL Y-14430.

14. The yeast strain of claim 1, wherein the strain is deficient in OCH1 α-1,6-mannosyltransferase activity.

15. The method according to claim 10, further comprising isolating the heterologous glycoprotein from the cultivated yeast.


SEQUENCE LISTING (15 sequences)

SEQ ID NO: 1 - protein, 404 aa, Komagataella phaffii
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 2 - protein, 403 aa, synthetic construct
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 3 - DNA, 21 bp, synthetic construct
  source 1..21: mol_type = other DNA
ttcaaaagaaggtcgcctggt

SEQ ID NO: 4 - DNA, 22 bp, synthetic construct
  source 1..22: mol_type = other DNA
agtcagccacgattctattggt

SEQ ID NO: 5 - DNA, 24 bp, synthetic construct
  source 1..24: mol_type = other DNA
tctctcctggaacattctctatcg

SEQ ID NO: 6 - DNA, 22 bp, synthetic construct
  source 1..22: mol_type = other DNA
agtcagccacgattctattggt

SEQ ID NO: 7 - DNA, 21 bp, synthetic construct
  source 1..21: mol_type = other DNA
gactggttccaattgacaagc

SEQ ID NO: 8 - DNA, 21 bp, synthetic construct
  source 1..21: mol_type = other DNA
gtcagtgcggtcatcatgcag

SEQ ID NO: 9 - DNA, 26 bp, synthetic construct
  source 1..26: mol_type = other DNA
caaatggcattctgacatcctcttga

SEQ ID NO: 10 - RNA, 20 nt, synthetic construct
  source 1..20: mol_type = other RNA
cgaacgtgtaccagaagtct

SEQ ID NO: 11 - protein, 359 aa, synthetic construct
GSQVQLQESGGGLVQTGGSLRLSCAFSGFTSDDYVIGWFRQAPGKGRQGVSCIRLSGGGTIYADSAKGRFTVSADNAKKTVYLQMTRLKPEDTAVYYCGAERYNVEGCGYDVAYWGKGTQVTVSSGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 25 aa, synthetic construct
SLLTEVETPIRNEWGCRCNDSSDSG

SEQ ID NO: 13 - protein, 25 aa, synthetic construct
SLLTEVETPTRNGWECRYSGSSDSG

SEQ ID NO: 14 - protein, 25 aa, synthetic construct
SLLTEVETPTRNEWGCRCSDSSDSG

SEQ ID NO: 15 - protein, 480 aa, Saccharomyces cerevisiae
MSRKLSHLIATRKSKTIVVTVLLIYSLLTFHLSNKRLLSQFYPSKDDFKQTLLPTTSHSQDINLKKQITVNKKKNQLHNLRDQLSFAFPYDSQAPIPQRVWQTWKVGADDKNFPSSFRTYQKTWSGSYSPDYQYSLISDDSIIPFLENLYAPVPIVIQAFKLMPGNILKADFLRYLLLFARGGIYSDMDTMLLKPIDSWPSQNKSWLNNIIDLNKPIPYKNSKPSLLSSDEISHQPGLVIGIEADPDRDDWSEWYARRIQFCQWTIQAKPGHPILRELILNITATTLASVQNPGVPVSEMIDPRFEEDYNVNYRHKRRHDETYKHSELKNNKNVDGSDIMNWTGPGIFSDIIFEYMNNVLRYNSDILLINPNLNKNDEEGSESATTPAKDVDNDTLSKSTRKFYKKISESLQSSNSMPWEFFSFLKEPVIVDDVMVLPITSFSPDVGQMGAQSSDDKMAFVKHMFSGSWKEDADKNAGHK